(19) United States
(12) Patent Application Publication
(10) Pub. No.: US 2023/0041334 A1
(43) Pub. Date: Feb. 9, 2023

(54) METHODS AND COMPOSITIONS FOR SMOKING CESSATION

(71) Applicants: BEHAVIORAL DIAGNOSTICS, LLC, Iowa City, IA (US); UNIVERSITY OF IOWA RESEARCH FOUNDATION, Iowa City, IA (US)

(72) Inventors: Eric A. HOFFMAN, Iowa City, IA (US); Albert Tao-man LEUNG, Ringoes, NJ (US); Alejandro Comellas, Iowa City, IA (US); Alicia GERKE, Iowa City, IA (US); Robert PHILIBERT, Iowa City, IA (US)

(73) Assignees: BEHAVIORAL DIAGNOSTICS, LLC, Iowa City, IA (US); UNIVERSITY OF IOWA RESEARCH FOUNDATION, Iowa City, IA (US)

(21) Appl. No.: 17/776,968

(22) PCT Filed: Nov. 13, 2020
(86) PCT No.: PCT/US2020/060529

§ 371 Date: May 13, 2022

Related U.S. Application Data
(60) Provisional application No. 62/935,456, filed on Nov. 14, 2019.
(60) Provisional application No. 62/961,949, filed on Jan. 16, 2020.

(51) Int. Cl. A61K 31/519; A61K 31/4162; A61K 31/5375; A61K 31/352; A61P 25/34

(52) U.S. Cl. CPC A61K 31/519; A61K 31/4162; A61K 31/5375; A61K 31/352; A61P 25/34

(57) ABSTRACT

This disclosure describes the use of a phosphodiesterase-5 (PDE5) inhibitor to reduce an individual's desire to smoke and/or frequency of smoking. In some embodiments, the PDE5 inhibitor is sildenafil (e.g., Viagra).

### CROSS REFERENCE TO RELATED APPLICATIONS

This application claims the benefit of priority under 35 U.S.C. 119(e) to U.S. Application No. 62/935,456 filed Nov. 14, 2019 and Application No. 62/961,949 filed Jan. 16, 2020, the entire contents of each of the foregoing are incorporated herein by reference.

### FEDERALLY SPONSORED RESEARCH OR DEVELOPMENT

This invention was made with government support under R01 HL130833 awarded by National Institutes of Health. The government has certain rights in the invention.

### TECHNICAL FIELD

This disclosure generally relates to methods and compositions to promote smoking cessation.

### BACKGROUND

Smoking is the most common preventable cause of morbidity and mortality in the United States. Therefore, there are extensive public policy measures in place to help ensure that people do not begin to smoke. Still, approximately 1 in 6 Americans begin this deadly habit, which, when initiated, is very difficult to stop. Even if smoking is stopped, inflammation persists, likely limiting a sense of improved well-being.

In order to help patients to stop smoking, scientists have devised a number of pharmacotherapies and behavioral interventions. Perhaps the best known are the three FDA approved pharmacotherapies: nicotine replacement (e.g., in the form of gum or a transdermal patch), bupropion (e.g., Wellbutrin®) and varenicline (e.g., Chantix®). However, behavioral therapies, including motivational interviewing and, more recently, incentive-based programming, have been shown to be effective in aiding smoking cessation. Still, despite the availability of these options, the effectiveness in standard clinical care is less than 10%, with many patients failing repeatedly. Therefore, there is a desperate need to develop new therapeutic approaches to address and improve smoking cessation.

### SUMMARY

The use of a phosphodiesterase-5 (PDE5) inhibitor to reduce an individual's urge to smoke is described herein.

In one aspect, a method of reducing an individual's desire to smoke and/or frequency of smoking is provided. In some embodiments, the method includes administering to the individual a phosphodiesterase-5 (PDE5) inhibitor or pharmaceutically acceptable salt thereof in an amount effective to reduce the individual's desire to smoke, reduce the frequency of smoking, or both.

In some embodiments, the PDE5 inhibitor is a compound of Formula I:

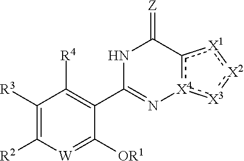

or a pharmaceutically acceptable salt thereof, wherein:

Z is O or S;

W is N or CR⁵;

X¹ and X² are each independently selected from N, NR⁶, and CR⁷;

X³ is N or CR⁸;

X⁴ is C or N;

R¹ is H, C₁₋₆ alkyl, or -L-O—(C₁₋₆ alkyl);

R² is H, C₁₋₆ alkyl, or C₄₋₁₀ cycloalkyl;

R³ is H, NO₂, C₁₋₆ alkyl, C₄₋₁₀ cycloalkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, C₄₋₁₀ cycloalkyl, or C(═O)(C₁₋₆ alkyl);

R⁵ is H, C₁₋₆ alkyl, or C₄₋₁₀ cycloalkyl;

R⁶ is H, C₁₋₆ alkyl, C₄₋₁₀ cycloalkyl, -L-O—(C₁₋₆ alkyl), -L-O—(C₄₋₁₀ cycloalkyl), -L-aryl, -L-hetAr¹, or -L-hetCyc¹;

R⁷ is H, C₁₋₆ alkyl, or C₄₋₁₀ cycloalkyl;

R⁸ is H, C₁₋₆ alkyl, or C₄₋₁₀ cycloalkyl;

R⁹ is C₁₋₆ alkyl, C₄₋₁₀ cycloalkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl);

L is absent or C₁₋₆ alkyl; and

the dashed lines can be single or double bonds.

In some embodiments of the method, the compound of Formula I is a compound of Formula Ia:

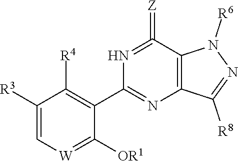

or a pharmaceutically acceptable salt thereof, wherein:

Z is O or S;

W is N or CR⁵;

R¹ is H, C₁₋₆ alkyl, or -L-O—(C₁₋₆ alkyl);

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, or C(═O)(C₁₋₆ alkyl);

R⁵ is H or C₁₋₆ alkyl;

R⁶ is H, C₁₋₆ alkyl, -L-O—(C₁₋₆ alkyl), -L-aryl, -L-hetAr¹, or -L-hetCyc¹;

R⁸ is H or C₁₋₆ alkyl;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, Z is O. In some embodiments, Z is S.

In some embodiments, W is CH.

In some embodiments, R¹ is H. In some embodiments, R¹ is C₁₋₃ alkyl. In some embodiments, R¹ is ethyl. In some embodiments, R¹ is propyl.

In some embodiments, R⁴ is H.

In some embodiments, R⁶ is C₁₋₃ alkyl. In some embodiments, R⁶ is methyl.

In some embodiments, R⁸ is C₁₋₃ alkyl. In some embodiments, R⁸ is propyl.

In some embodiments of the method, the compound of Formula I is a compound of Formula Ib:

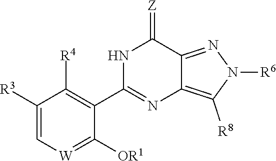

or a pharmaceutically acceptable salt thereof, wherein:

Z is O or S;

W is N or CR⁵;

R¹ is H, C₁₋₆ alkyl, or -L-O—(C₁₋₆ alkyl);

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, or C(═O)(C₁₋₆ alkyl);

R⁵ is H or C₁₋₆ alkyl;

R⁶ is H, C₁₋₆ alkyl, -L-O—(C₁₋₆ alkyl), -L-aryl, -L-hetAr¹, or -L-hetCyc¹;

R⁸ is H or C₁₋₆ alkyl;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, Z is O.

In some embodiments, W is CH. In some embodiments, W is N.

In some embodiments, R¹ is C₁₋₆ alkyl. In some embodiments, R¹ is ethyl, propyl, or butyl. In some embodiments, R¹ is —(C₁₋₆ alkyl)-O—(C₁₋₆ alkyl).

In some embodiments, R⁴ is H. In some embodiments, R⁴ is C(═O)(C₁₋₆ alkyl). In some embodiments, C₁₋₆ alkyl is methyl.

In some embodiments, R⁶ is C₁₋₃ alkyl. In some embodiments, R⁶ is —(C₁₋₆ alkyl)-O—(C₁₋₆ alkyl). In some embodiments, R⁶ is phenyl. In some embodiments, R⁶ is —(C₁₋₆ alkyl)-hetAr¹. In some embodiments, hetAr¹ is pyridine. In some embodiments, R⁶ is hetCyc¹ optionally substituted with C₁₋₆ alkyl. In some embodiments, R⁶ is piperidine or azetidine substituted with C₁₋₃ alkyl.

In some embodiments, R⁸ is C₁₋₃ alkyl. In some embodiments, R⁸ is ethyl.

In some embodiments of the method, the compound of Formula I is a compound of

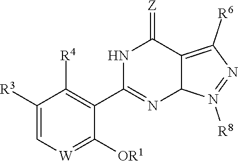

or a pharmaceutically acceptable salt thereof, wherein:

Z is O or S;

W is N or CR⁵;

R¹ is H, C₁₋₆ alkyl, or -L-O—(C₁₋₆ alkyl);

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, or C(═O)(C₁₋₆ alkyl);

R⁵ is H or C₁₋₆ alkyl;

R⁶ is H, C₁₋₆ alkyl, -L-O—(C₁₋₆ alkyl), -L-aryl, -L-hetAr¹, or -L-hetCyc¹;

R⁸ is H or C₁₋₆ alkyl;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, Z is O.

In some embodiments, W is CH.

In some embodiments, R¹ is C₁₋₃ alkyl. In some embodiments, R¹ is ethyl.

In some embodiments, R⁴ is H.

In some embodiments, R⁶ is C₁₋₃ alkyl. In some embodiments, R⁶ is methyl.

In some embodiments, R⁸ is C1-3 alkyl. In some embodiments, R⁸ is propyl.

In some embodiments of the method, the compound of Formula I is a compound of Formula Id:

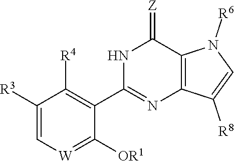

or a pharmaceutically acceptable salt thereof, wherein:

Z is O or S;

W is N or CR⁵;

R¹ is H, C₁₋₆ alkyl, or -L-O—(C₁₋₆ alkyl);

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, or C(═O)(C₁₋₆ alkyl);

R⁵ is H or C₁₋₆ alkyl;

R⁶ is H, C₁₋₆ alkyl, -L-O—(C₁₋₆ alkyl), -L-aryl, -L-hetAr¹, or -L-hetCyc¹;

R⁸ is H or C₁₋₆ alkyl;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, Z is O.

In some embodiments, W is CH.

In some embodiments, R¹ is C₁₋₃ alkyl. In some embodiments, R¹ is propyl.

In some embodiments, R⁴ is H.

In some embodiments, R⁶ is C₁₋₃ alkyl. In some embodiments, R⁶ is ethyl.

In some embodiments, R⁸ is C₁₋₃ alkyl. In some embodiments, R⁸ is propyl.

In some embodiments of the method, the compound of Formula I is a compound of Formula Ie:

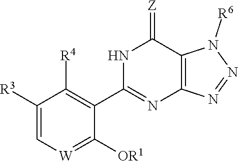

or a pharmaceutically acceptable salt thereof, wherein:

Z is O or S;

W is N or CR⁵;

R¹ is H, C₁₋₆ alkyl, or -L-O—(C₁₋₆ alkyl);

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, or C(═O)(C₁₋₆ alkyl);

R⁵ is H or C₁₋₆ alkyl;

R⁶ is H, C₁₋₆ alkyl, -L-O—(C₁₋₆ alkyl), -L-aryl, -L-hetAr¹, or -L-hetCyc¹;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, Z is O.

In some embodiments, W is CH.

In some embodiments, R¹ is C₁₋₃ alkyl. In some embodiments, R¹ is propyl.

In some embodiments, R⁴ is H.

In some embodiments, R⁶ is H.

In some embodiments of any one of the compounds of Formula I and Formulas Ia-Ie, R³ is selected from

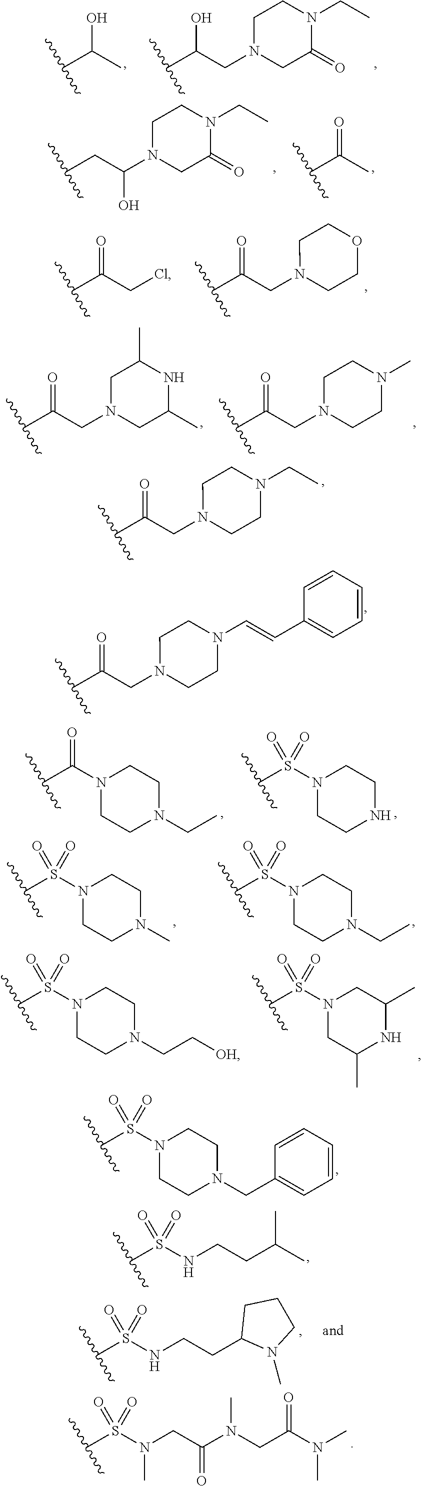

In some embodiments of the method, the PDE5 inhibitor is a compound of Formula II:

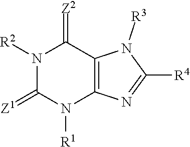

or a pharmaceutically acceptable salt thereof, wherein:

Z¹ is O or S;

Z² is O or S;

R¹ is H, C₁₋₆ alkyl, C₄₋₁₀ cycloalkyl, or C₁₋₆ hydroxyalkyl;

R² is H, C₁₋₆ alkyl, C₄₋₁₀ cycloalkyl, or C₁₋₆ hydroxyalkyl;

R³ is H, C₁₋₆ alkyl, C₄₋₁₀ cycloalkyl, C₁₋₆ alkyl(hetCyc¹), C₁₋₆ alkyl(hetAr¹), or C₁₋₆ alkyl(aryl), wherein any C₁₋₆ alkyl is optionally substituted with one or more hydroxy and halogen, and aryl is optionally substituted with one or more substituents independently selected from halogen, C₁₋₆ alkyl, amino, cyano, C₁₋₆ alkoxy, and hydroxy;

R⁴ is H, C₁₋₆ alkyl, C₄₋₁₀ cycloalkyl, or NR′R″, wherein R′ and R″ are each independently selected from H, C₁₋₆ alkyl, C₄₋₁₀ cycloalkyl, hetCyc¹, hetAr¹, aryl, C₁₋₆ alkyl(hetCyc¹), C₁₋₆ alkyl(hetAr¹), and C₁₋₆ alkyl(aryl), and wherein any C₁₋₆ alkyl or C₄₋₁₀ cycloalkyl is optionally substituted with one or more hydroxy and halogen, and aryl is optionally substituted with one or more substituents independently selected from halogen, C₁₋₆ alkyl, amino, cyano, C₁₋₆ alkoxy, and hydroxy;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy; and

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

In some embodiments, Z¹ and Z² are each O.

In some embodiments, R¹ is C₁₋₃ hydroxyalkyl.

In some embodiments, R² is C₁₋₃ alkyl. In some embodiments, R² is ethyl.

In some embodiments, R³ is C₁₋₃ alkyl(aryl) optionally substituted with one or two substituents independently selected from halogen and C₁₋₆ alkoxy.

In some embodiments, R⁴ is NR′R″. In some embodiments, R′ is H. In some embodiments, R″ is C₄₋₁₀ cycloalkyl optionally substituted with hydroxy. In some embodiments, C₄₋₁₀ cycloalkyl is cyclopentyl.

In some embodiments of the method, the PDE5 inhibitor is a compound of Formula III:

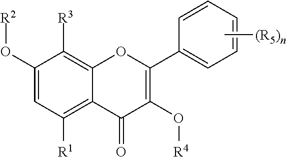

or a pharmaceutically acceptable salt thereof, wherein:

R¹ is H, hydroxy, C₁₋₆ alkyl, C₄₋₁₀ cycloalkyl, or C₁₋₆ hydroxyalkyl;

R² is H, C₁₋₆ alkyl, C₄₋₁₀ cycloalkyl, or hetCyc¹;

R³ is H, C₁₋₆ alkyl, C₄₋₁₀ cycloalkyl, or C₂₋₁₀ alkenyl;

R⁴ is H, C₁₋₆ alkyl, C₄₋₁₀ cycloalkyl, or hetCyc¹;

R⁵ is H, C₁₋₆ alkyl, C₄₋₁₀ cycloalkyl, or C₁₋₆ alkoxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

n is 0 to 5.

In some embodiments, R¹ is hydroxy.

In some embodiments, R² is hetCyc¹ optionally substituted with one to four substituents independently selected from hydroxy and C₁₋₃ hydroxyalkyl. In some embodiments, R² is tetrahydropyran substituted with one to four substituents independently selected from hydroxy and C₁₋₃ hydroxyalkyl.

In some embodiments, R³ is C₂₋₁₀ alkenyl. In some embodiments, R³ is C5 alkenyl.

In some embodiments, R⁴ is hetCyc¹ optionally substituted with one to four substituents independently selected from hydroxy and C₁₋₃ alkyl. In some embodiments, R⁴ is tetrahydropyran substituted with one to four substituents independently selected from hydroxy and C₁₋₃ alkyl.

In some embodiments, R⁵ is C₁₋₆ alkoxy. In some embodiments, R⁵ is methoxy.

In some embodiments, n is 1.

In some embodiments of the method, the PDE5 inhibitor is a compound of Formula IV:

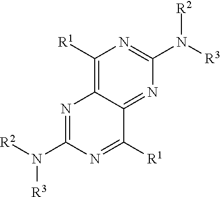

or a pharmaceutically acceptable salt thereof, wherein:

R¹ is H, amino, C₁₋₆ alkyl, C₁₋₆ hydroxyalkyl, C₁₋₆ alkoxy, C₄₋₁₀ cycloalkyl, or hetCyc¹;

R² and R³ are each independently selected from H, C₁₋₆ alkyl, C₁₋₆ hydroxyalkyl, C₁₋₆ alkoxy, C₄₋₁₀ cycloalkyl, and hetCyc¹; and

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

In some embodiments, R¹ is hetCyc¹. In some embodiments, hetCyc¹ is piperidine.

In some embodiments, R² is C₁₋₃ hydroxyalkyl.

In some embodiments, R³ is C₁₋₃ hydroxyalkyl.

In some embodiments, R² and R³ are each C₁₋₃ hydroxyalkyl.

In some embodiments of the method, the PDE5 inhibitor is a compound of Formula V:

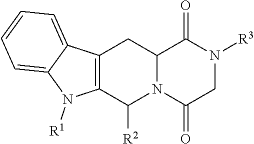

or a pharmaceutically acceptable salt thereof, wherein:

R¹, R², and R³ are each independently selected from H, C₁₋₆ alkyl, C₁₋₆ hydroxyalkyl, C₁₋₆ alkoxy, C₄₋₁₀ cycloalkyl, and hetCyc¹; and

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

In some embodiments, R¹ is H.

In some embodiments, R² is hetCyc¹. In some embodiments, hetCyc¹ is 1,3-benzodioxole.

In some embodiments, R³ is C₁₋₃ alkyl. In some embodiments, R³ is methyl.

In some embodiments of the method, the PDE5 inhibitor is a compound of Formula VI:

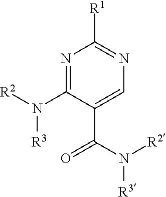

or a pharmaceutically acceptable salt thereof, wherein:

R¹ is H, C₁₋₆ alkyl, C₁₋₆ hydroxyalkyl, C₁₋₆ alkoxy, C₄₋₁₀ cycloalkyl, or hetCyc¹;

R², R^{2′}, R³ and R^{3′} are each independently selected from H, C₁₋₆ alkyl, C₁₋₆ hydroxyalkyl, C₁₋₆ alkoxy, C₄₋₁₀ cycloalkyl, aryl, hetCyc¹, hetAr¹, C₁₋₆ alkyl(aryl), C₁₋₆ alkyl(hetCyc¹), and C₁₋₆ alkyl(hetAr¹), wherein aryl is optionally substituted with halogen, C₁₋₆ alkyl, C₁₋₆ alkoxy, amino, cyano, hydroxy, and C₁₋₆ hydroxyalkyl;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy.

In some embodiments, R¹ is hetCyc¹ optionally substituted with C₁₋₃ hydroxyalkyl. In some embodiments, hetCyc¹ is pyrrolidine.

In some embodiments, R² and R^{2′} are each H.

In some embodiments, R³ is C₁₋₃ alkyl(aryl) substituted with one or two substituents selected from halogen and C₁₋₃ alkoxy.

In some embodiments, R^{3′} is C₁₋₃ alkyl(hetAr¹). In some embodiments, hetAr¹ is pyrimidine.

In some embodiments of the method, the PDE5 inhibitor is a compound of Formula VII:

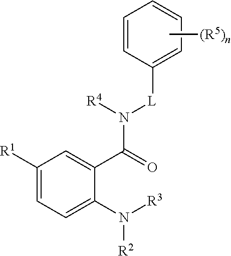

or a pharmaceutically acceptable salt thereof, wherein:

R¹ is H, amino, nitro, C₁₋₆ alkyl, C₁₋₆ hydroxyalkyl, C₁₋₆ alkoxy, C₄₋₁₀ cycloalkyl, or hetCyc¹;

R², R³, and R⁴ are each independently selected from H, C₁₋₆ alkyl, C₁₋₆ hydroxyalkyl, C₁₋₆ alkoxy, C₄₋₁₀ cycloalkyl, and hetCyc¹;

R⁵ is H, amino, nitro, C₁₋₆ alkyl, C₁₋₆ hydroxyalkyl, C₁₋₆ alkoxy, C₄₋₁₀ cycloalkyl;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl);

L is —C₁₋₆ alkyl- or —C₁₋₆ alkoxy-; and

n is 0 to 5.

In some embodiments, R¹ is nitro.

In some embodiments, R² is H.

In some embodiments, R³ is C1-3 hydroxyalkyl.

In some embodiments, R⁴ is H.

In some embodiments, R⁵ is C₁₋₆ alkoxy. In some embodiments, R⁵ is methoxy.

In some embodiments, n is 1.

In some embodiments, L is —C₁₋₃ alkyl-.

In some embodiments of the method, the PDE5 inhibitor is a compound selected from the compounds of Table 1 and pharmaceutically acceptable salts thereof.

In some embodiments of the method, the PDE5 inhibitor is a compound selected from

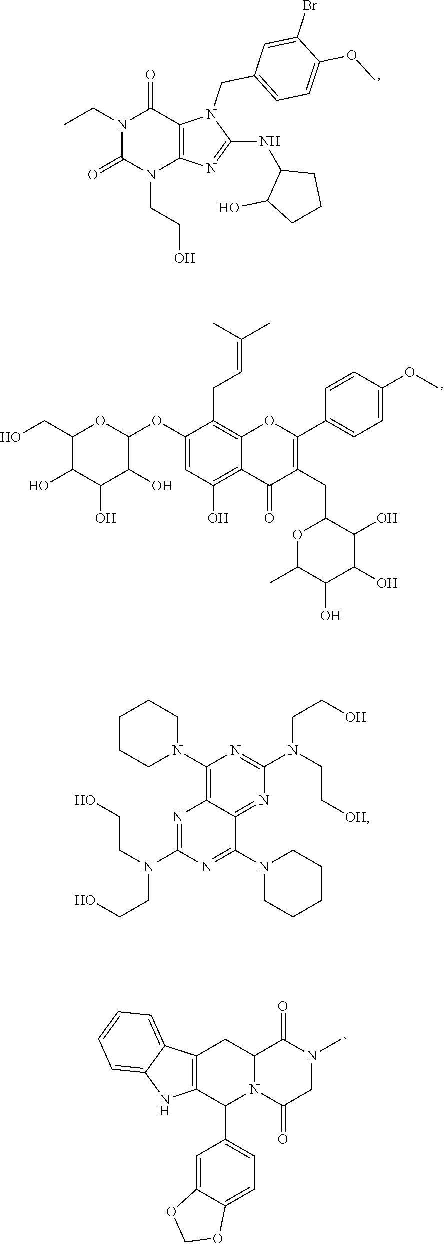 [SMILES: CCn1c(=O)c2c(nc(NC3CCCC3O)n2Cc2ccc(OC)c(Br)c2)n(CCO)c1=O ; COc1ccc(-c2oc3c(CC=C(C)C)c(OC4OC(CO)C(O)C(O)C4O)cc(O)c3c(=O)c2CC2OC(C)C(O)C(O)C2O)cc1 ; OCCN(CCO)c1nc(N2CCCCC2)c2nc(N(CCO)CCO)nc(N3CCCCC3)c2n1 ; CN1CC(=O)N2C(Cc3c([nH]c4ccccc34)C2c2ccc3c(c2)OCO3)C1=O]

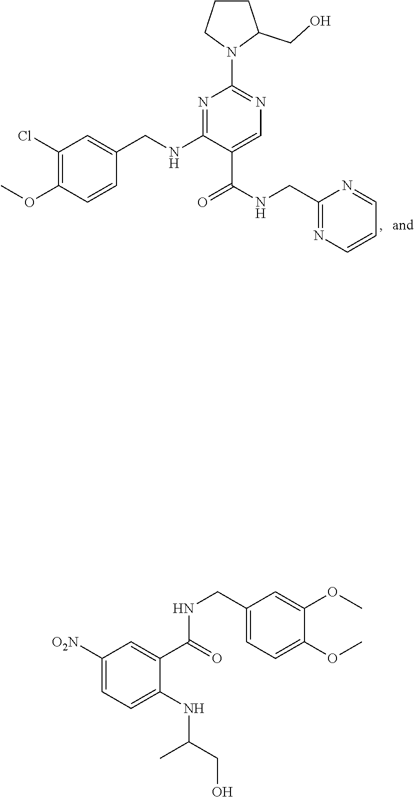 [SMILES: COc1ccc(CNc2nc(N3CCCC3CO)ncc2C(=O)NCc2ncccn2)cc1Cl ; COc1ccc(CNC(=O)c2cc([N+](=O)[O-])ccc2NC(C)CO)cc1OC]

and pharmaceutically acceptable salts thereof.

In some embodiments of the method, the PDE5 inhibitor is selected from

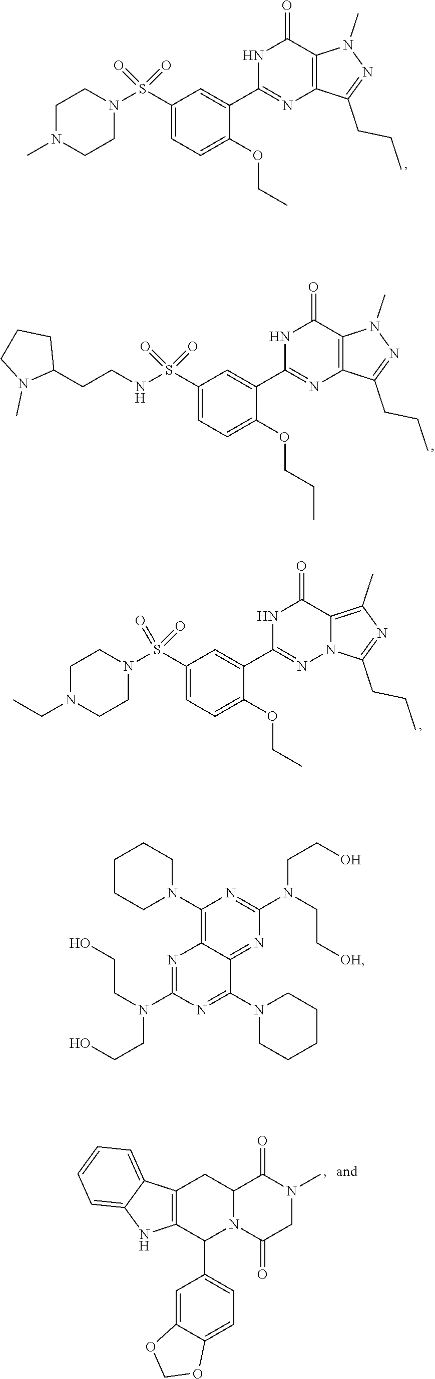

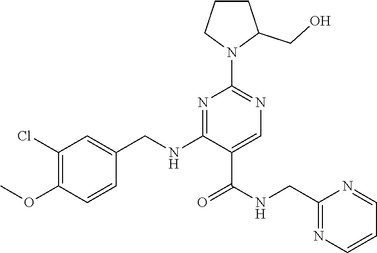 [SMILES: COc1ccc(CNc2nc(N3CCCC3CO)ncc2C(=O)NCc2ncccn2)cc1Cl]

and pharmaceutically acceptable salts thereof.

In some embodiments of the method, the PDE5 inhibitor is sildenafil:

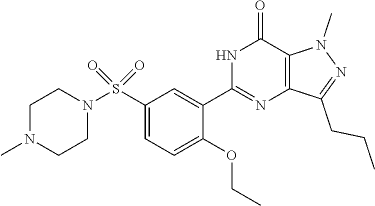 [SMILES: CCCc1nn(C)c2c(=O)[nH]c(-c3cc(S(=O)(=O)N4CCN(C)CC4)ccc3OCC)nc12]

or a pharmaceutically acceptable salt thereof.

In some embodiments of the method, the PDE5 inhibitor or pharmaceutically acceptable salt thereof is administered to the individual at a dose of about 1 mg/day to about 150 mg/day. In some embodiments, the effective amount of the PDE5 inhibitor or pharmaceutically acceptable salt thereof is from about 1 mg/day to about 150 mg/day.

In some embodiments, the PDE5 inhibitor or pharmaceutically acceptable salt thereof is formulated for oral administration. In some embodiments, the PDE5 inhibitor or pharmaceutically acceptable salt thereof is formulated for extended/delayed release.

In another aspect, methods of reducing an individual's desire to smoke and/or frequency of smoking are provided. Such methods typically include administering to the individual an amount of sildenafil (Viagra®), a derivative of sildenafil, an enantiomer of sildenafil, an active metabolite of sildenafil, or a pharmaceutically acceptable salt of sildenafil effective to reduce the individual's desire to smoke and/or frequency of smoking.

In some embodiments, the sildenafil or derivative, enantiomer or pharmaceutically acceptable salt thereof is administered to the individual at a dose of about 1 mg/day to about 150 mg/day. In some embodiments, the effective amount of the sildenafil or derivative, enantiomer, metabolite, or pharmaceutically acceptable salt thereof is from about 1 mg/day to about 150 mg/day.

In some embodiments, the sildenafil or derivative, enantiomer, metabolite, or pharmaceutically acceptable salt thereof is formulated for oral administration. In some embodiments, the sildenafil or derivative, enantiomer, metabolite, or pharmaceutically acceptable salt thereof is formulated for extended/delayed release.

In some embodiments, the methods described herein further include co-administering to the individual nicotine replacement (e.g., in the form of gum or a transdermal patch), bupropion (e.g., Wellbutrin®), tadalafil (e.g., Cialis®), vardenafil (e.g., Levitra®), and varenicline (e.g., Chantix®), or derivatives, enantiomers, metabolites, or pharmaceutically acceptable salts thereof. In some embodiments, the methods described herein further include applying motivational interviewing, incentive based programming, or other psychological technique(s) to the individual.

In some embodiments, the individual, following administration for a period of time, exhibits an improvement in the lung diffusing capacity for carbon monoxide (DLCO). In some embodiments, the individual, following administration for a period of time, exhibits tissue re-perfusion. In some embodiments, the individual, following administration for a period of time, exhibits a reduction in parenchymal inflammation (or lung density).

In another aspect, articles of manufacture are provided. Such articles of manufacture typically include at least one dose of a PDE5 inhibitor effective to reduce an individual's desire to smoke and/or frequency of smoking; and at least one dose of a nicotine replacement (e.g., in the form of gum or a transdermal patch), bupropion (e.g., Wellbutrin®) tadalafil (e.g., Cialis®), vardenafil (e.g., Levitra®), or varenicline (e.g., Chantix®), or derivatives, enantiomers, metabolites, or pharmaceutically acceptable salts thereof effective to reduce an individual's desire to smoke and/or frequency of smoking. In some embodiments, the PDE5 inhibitor is sildenafil (Viagra®), a derivative of sildenafil, an enantiomer of sildenafil, an active metabolite of sildenafil, or a pharmaceutically acceptable salt of sildenafil.

In some embodiments, the at least one dose of the PDE5 inhibitor comprises about 1 mg to about 150 mg. In some embodiments, the PDE5 inhibitor is formulated for oral administration. In some embodiments, the PDE5 inhibitor is formulated for extended/delayed release. In some embodiments, the PDE5 inhibitor is sildenafil (Viagra®), a derivative of sildenafil, an enantiomer of sildenafil, an active metabolite of sildenafil, or a pharmaceutically acceptable salt of sildenafil.

In still another aspect, articles of manufacture are provided. Such articles of manufacture typically include at least one dose of a PDE5 inhibitor, where the at least one dose includes about 1 mg to about 40 mg of the PDE5 inhibitor. In some embodiments, the PDE5 inhibitor is sildenafil (Viagra®), a derivative of sildenafil, an enantiomer of sildenafil, an active metabolite of sildenafil, or a pharmaceutically acceptable salt of sildenafil.

In some embodiments, the methods described herein further include co-administration of at least one dose of a nicotine replacement (e.g., in the form of gum or a transdermal patch), bupropion (e.g., Wellbutrin®) tadalafil (e.g., Cialis®), vardenafil (e.g., Levitra®), or varenicline (e.g., Chantix®), or derivatives, enantiomers, metabolites, or pharmaceutically acceptable salts thereof.

In some embodiments, the PDE5 inhibitor is formulated for oral administration. In some embodiments, the PDE5 inhibitor is formulated for extended/delayed release. In some embodiments, the PDE5 inhibitor is sildenafil (Viagra®), a derivative of sildenafil, an enantiomer of sildenafil, an active metabolite of sildenafil, or a pharmaceutically acceptable salt of sildenafil.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DETAILED DESCRIPTION

Sildenafil (e.g., Viagra®), which is a phosphodiesterase-5 (PDE5) inhibitor, was originally developed as a pulmonary anti-hypertensive, but is better known for its use in treating erectile dysfunction. The structure of sildenafil is

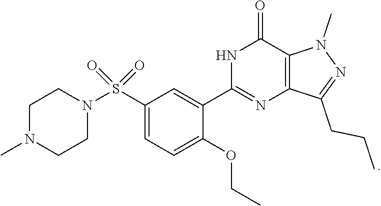 [SMILES: CCCc1nn(C)c2c(=O)[nH]c(-c3cc(S(=O)(=O)N4CCN(C)CC4)ccc3OCC)nc12]

Other known phosphodiesterase-5 inhibitors include, without limitation, tadalafil (e.g., Cialis®), vardenafil (e.g., Levitra®), udenafil, avanafil, and dipyridamole.

The present inventors have surprisingly found that phosphodiesterase-5 inhibitors can significantly reduce an individual's desire to smoke and/or frequency of smoking, thereby increasing the likelihood that an individual will stop smoking. A reduction in a desire to smoke and/or frequency of smoking is typically self-reported, but can be reflected in the number of cigarettes and/or frequency of cigarettes smoked and/or the level of cotinine found in the individual. Generally, an individual is considered to have stopped smoking when they have not smoked a cigarette for a period of time (e.g., at least one month, at least two months, at least six months, at least one year, etc.).

The likelihood that an individual will stop smoking can be further increased by co-administering, along with a phosphodiesterase-5 inhibitor, a form of nicotine replacement (e.g., nicotine gum, a nicotine transdermal patch) and/or another compound known to reduce the desire to smoke and/or frequency of smoking (e.g., bupropion (e.g., Wellbutrin®), varenicline (e.g., Chantix®)). In addition, motivational interviewing, incentive based programming, or other psychological technique(s) can be provided to the individual in order to increase the likelihood that they will cease smoking.

Accordingly, the present disclosure provides compounds and methods useful for reducing an individual's desire to smoke, the frequency of smoking, or both. The methods include administering to the individual an amount of a PDE5 inhibitor that is effective to reduce the individual's desire to smoke, the frequency of smoking, or both.

### Definitions

The term “substituted,” as used herein, means that any one or more hydrogens on the designated atom, usually a carbon, oxygen, or nitrogen atom, is replaced with a selection from the indicated group, provided that the designated atom's normal valency is not exceeded, and that the substitution results in a stable compound. When a substituent is keto or oxo (i.e., ═O), then 2 hydrogens on the atom are replaced.

As used herein, “alkyl” is intended to include both branched and straight-chain saturated aliphatic hydrocarbon groups having the specified number of carbon atoms. For example, C₁₋₄ alkyl is intended to include C₁, C₂, C₃, and C₄. C₁₋₆ alkyl is intended to include C₁, C₂, C₃, C₄, C₅, and C₆ alkyl groups and C₁₋₈ alkyl is intended to include C₁, C₂, C₃, C₄, C₅, C₆, C₇, and C₈. Some examples of alkyl include, but are not limited to, methyl, ethyl, n-propyl, iso-propyl, n-butyl, s-butyl, t-butyl, n-pentyl, s-pentyl, n-hexyl, n-heptyl, and n-octyl.

As used herein, “alkenyl” is intended to include hydrocarbon chains of either straight or branched configuration and one or more unsaturated carbon-carbon bond that can occur in any stable point along the chain, such as ethenyl and propenyl. For example, C₂₋₆ alkenyl is intended to include C₂, C₃, C₄, C₅, and C₆ alkenyl groups and C₂₋₈ alkenyl is intended to include C₂, C₃, C₄, C₅, C₆, C₇, and C₈ alkenyl groups.

As used herein, “cycloalkyl” is intended to include saturated or unsaturated nonaromatic ring groups, such as cyclopropyl, cyclobutyl, cyclopentyl, or cyclohexyl. For example, the term “C₃₋₈ cycloalkyl” is intended to include C₃, C₄, C₅, C₆, C₇, and C₈ cycloalkyl groups. Cycloalkyls may include multiple spiro- or fused or bridged rings. For example, cycloalkyl can include, but is not limited to, spiro butyl, pentyl, hexyl, heptyl, octyl, nonyl, or decyl groups, bicyclo butyl, pentyl, hexyl, heptyl, octyl, nonyl, or decyl groups, adamantyl groups, and norbornyl groups.

As used herein, the term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, or Se), unless specified otherwise. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, tetrahyrofuranyl, oxiranyl, azetidinyl, oxetanyl, thietanyl, 1,2,3,6-tetrahydropyridinyl, tetrahydropyranyl, dihydropyranyl, pyranyl, morpholinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl and the like.

As used herein, “amine” or “amino” refers to unsubstituted —NH₂ unless otherwise specified.

As used herein, “halo” or “halogen” refers to fluoro, chloro, bromo, and iodo substituents.

As used herein, “alkoxy” refers to an alkyl group as defined above with the indicated number of carbon atoms attached through an oxygen bridge. C₁₋₆ alkoxy, is intended to include C₁, C₂, C₃, C₄, C₅, and C₆ alkoxy groups. C₁₋₈ alkoxy, is intended to include C₁, C₂, C₃, C₄, C₅, C₆, C₇, and C₈ alkoxy groups. Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy.

As used herein, “aryl” includes groups with aromaticity, including “conjugated,” or multicyclic systems with at least one aromatic ring and do not contain any heteroatom in the ring structure. Aryl may be monocyclic or polycyclic (e.g., having 2, 3 or 4 fused rings). The term “Cₙ₋ₘ aryl” refers to an aryl group having from n to m ring carbon atoms. In some embodiments, aryl groups have from 6 to 10 carbon atoms. In some embodiments, the aryl group is phenyl or naphthyl.

As used herein, the term “heteroaryl ring” is intended to mean a stable 5, 6, 7, 8, 9, 10, 11, or 12-membered monocyclic or bicyclic aromatic ring which consists of carbon atoms and one or more heteroatoms, e.g., 1 or 1-2 or 1-3 or 1-4 or 1-5 or 1-6 heteroatoms, independently selected from nitrogen, oxygen, and sulfur. In the case of bicyclic aromatic heterocyclic or heterocycle or heteroaryl rings, only one of the two rings needs to be aromatic (e.g., 2,3-dihydroindole), though both can be (e.g., quinoline). The second ring can also be fused or bridged as defined above for heterocycles. The nitrogen atom can be substituted or unsubstituted (i.e., N or NR wherein R is H or another substituent, as defined). The nitrogen and sulfur heteroatoms can optionally be oxidized (i.e., N→O and S(O)ₚ, wherein p=1 or 2).

Examples of aromatic heterocycles, aromatic heterocyclics or heteroaryls include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, benzooxadiazoly, carbazolyl, 4aH-carbazolyl, carbolinyl, cinnolinyl, furazanyl, imidazolyl, imidazolonyl, 1H-indazolyl, indolizinyl, indolyl, 3H-indolyl, isobenzofuranyl, isochromanyl, isoindazolyl, isoindolyl, isoquinolinyl, isothiazolyl, isoxazolyl, methylbenztriazolyl, methylfuranyl, methylimidazolyl, methylthiazolyl, naphthyridinyl, oxadiazolyl, 1,2,3-oxadiazolyl, 1,2,4-oxadiazolyl, 1,2,5-oxadiazolyl, 1,3,4-oxadiazolyl, oxazolyl, phenanthridinyl, phenanthrolinyl, phenazinyl, phenothiazinyl, phenoxathinyl, phenoxazinyl, phthalazinyl, pteridinyl, purinyl, pyrazinyl, pyrazolyl, pyridazinyl, pyridooxazolyl, pyridoimidazolyl, pyridothiazolyl, pyridinyl, pyridinonyl, pyridyl, pyrimidinyl, 2H-pyrrolyl, pyrrolyl, quinazolinyl, quinolinyl, 4H-quinolizinyl, quinoxalinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, triazolopyrimidinyl, 1,2,3-triazolyl, 1,2,4-triazolyl, 1,2,5-triazolyl, and 1,3,4-triazolyl.

The term “hydroxyalkyl” means an alkyl group as defined above, where the alkyl group is substituted with one or more OH groups. Examples of hydroxyalkyl groups include HO—CH₂—, HO—CH₂—CH₂— and CH₃—CH(OH)—.

The term “cyano” as used herein means a substituent having a carbon atom joined to a nitrogen atom by a triple bond, i.e., C≡N.

As used herein, “oxo” is means a “═O” group.

As used herein, the phrase “pharmaceutically acceptable” refers to those compounds or tautomers thereof, or salts thereof, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds or tautomers thereof, wherein the parent compound or a tautomer thereof, is modified by making of the acid or base salts thereof of the parent compound or a tautomer thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound, or a tautomer thereof, formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, and toluene sulfonic.

The pharmaceutically acceptable salts of the present disclosure can be synthesized from the parent compound or a tautomer thereof that contains a basic or acidic moiety by conventional chemical methods. Generally, such pharmaceutically acceptable salts can be prepared by reacting the free acid or base forms of these compounds or tautomers thereof with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., USA, p. 1445 (1990).

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s).

Throughout the description, where compositions are described as having, including, or comprising specific components, or where processes are described as having, including, or comprising specific process steps, it is contemplated that compositions of the present disclosure also consist essentially of, or consist of, the recited components, and that the processes of the present disclosure also consist essentially of, or consist of, the recited processing steps. Further, it should be understood that the order of steps or order for performing certain actions are immaterial so long as the invention remains operable. Moreover, two or more steps or actions can be conducted simultaneously.

PDE5 Inhibitors

The present application provides a method of reducing an individual's desire to smoke, the frequency of smoking, or both, that includes administering to the individual an amount of a PDE5 inhibitor that is effective to reduce the individual's desire to smoke, the frequency of smoking, or both. In some embodiments, the PDE5 inhibitor is a compound of Formula I:

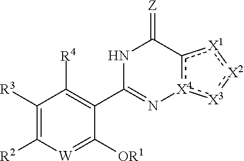

and pharmaceutically acceptable salts thereof, wherein:

Z is O or S;

W is N or CR⁵;

X¹ and X² are each independently selected from N, NR⁶, and CR⁷;

X³ is N or CR⁸;

X⁴ is C or N;

R¹ is H, C₁₋₆ alkyl, or -L-O—(C₁₋₆ alkyl);

R² is H, C₁₋₆ alkyl, or C₄₋₁₀ cycloalkyl;

R³ is H, NO₂, C₁₋₆ alkyl, C₄₋₁₀ cycloalkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, C₄₋₁₀ cycloalkyl, or C(═O)(C₁₋₆ alkyl);

R⁵ is H, C₁₋₆ alkyl, or C₄₋₁₀ cycloalkyl;

R⁶ is H, C₁₋₆ alkyl, C₄₋₁₀ cycloalkyl, -L-O—(C₁₋₆ alkyl), -L-O—(C₄₋₁₀ cycloalkyl), -L-aryl, -L-hetAr¹, or -L-hetCyc¹;

R⁷ is H, C₁₋₆ alkyl, or C₄₋₁₀ cycloalkyl;

R⁸ is H, C₁₋₆ alkyl, or C₄₋₁₀ cycloalkyl;

R⁹ is C₁₋₆ alkyl, C₄₋₁₀ cycloalkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl);

L is absent or C₁₋₆ alkyl; and

the dashed lines can be single or double bonds.

In some embodiments, Z is O. In some embodiments, Z is S.

In some embodiments, W is N. In some embodiments, W is CR⁵. In some embodiments, R⁵ is H. In some embodiments, R⁵ is C₁₋₆ alkyl. In some embodiments, R⁵ is C₄₋₁₀ cycloalkyl.

In some embodiments, X¹ is N.

In some embodiments, X¹ is NR⁶. In some embodiments, R⁶ is H. In some embodiments, R⁶ is C₁₋₆ alkyl. In some embodiments, R⁶ is methyl, ethyl, or propyl. In some embodiments, R⁶ is methyl. In some embodiments, R⁶ is C₄₋₁₀ cycloalkyl. In some embodiments, R⁶ is -L-O—(C₁₋₆ alkyl). In some embodiments, R⁶ is -L-O—(C₄₋₁₀ cycloalkyl). In some embodiments, R⁶ is -L-aryl. In some embodiments, R⁶ is -L-hetAr¹. In some embodiments, R⁶ is -L-hetCyc¹. In some embodiments, L is absent. In some embodiments, L is C₁₋₆ alkyl.

In some embodiments, X¹ is CR⁷. In some embodiments, R⁷ is H. In some embodiments, R⁷ is C₁₋₆ alkyl. In some embodiments, R⁷ is methyl, ethyl, or propyl. In some embodiments, R⁷ is C₄₋₁₀ cycloalkyl.

In some embodiments, X² is N.

In some embodiments, X² is NR⁶. In some embodiments, R⁶ is H. In some embodiments, R⁶ is C₁₋₆ alkyl. In some embodiments, R⁶ is C₄₋₁₀ cycloalkyl. In some embodiments, R⁶ is -L-O—(C₁₋₆ alkyl). In some embodiments L is (C₁₋₆ alkyl). In some embodiments, R⁶ is methoxyethyl. In some embodiments, R⁶ is -L-O—(C₄₋₁₀ cycloalkyl). In some embodiments, R⁶ is -L-aryl. In some embodiments, L is absent. In some embodiments, R⁶ is -L-hetAr¹. In some embodiments, hetAr¹ is pyridine. In some embodiments, R⁶ is -L-hetCyc¹. In some embodiments, L is absent. In some embodiments, hetCyc¹ is azetidine or piperidine optionally substituted with C₁₋₆ alkyl. In some embodiments, hetCyc¹ is ethylazetidine, isopropylazetidine, or methylpiperidine.

In some embodiments, X² is CR⁷. In some embodiments, R⁷ is H. In some embodiments, R⁷ is C₁₋₆ alkyl. In some embodiments, R⁷ is methyl, ethyl, or propyl. In some embodiments, R⁷ is C₄₋₁₀ cycloalkyl.

In some embodiments, X³ is N. In some embodiments, X³ is CR⁸. In some embodiments, R⁸ is H. In some embodiments, R⁸ is C₁₋₆ alkyl. In some embodiments, R⁸ is methyl, ethyl, or propyl. In some embodiments, R⁸ is propyl. In some embodiments, R⁸ is C₄₋₁₀ cycloalkyl.

In some embodiments, X⁴ is C. In some embodiments, X⁴ is N.

In some embodiments, R¹ is H. In some embodiments, R¹ is C₁₋₆ alkyl. In some embodiments, R¹ is ethyl, propyl, butyl, or isobutyl. In some embodiments, R¹ is ethyl. In some embodiments, R¹ is -L-O—(C₁₋₆ alkyl). In some embodiments, C₁₋₆ alkyl is methyl. In some embodiments, L is C₁₋₆ alkyl. In some embodiments, L is ethyl or isopropyl. In some embodiments, R¹ is 2-methoxyethoxy or (1-methoxypropan-2-yl)oxy.

In some embodiments, R² is H. In some embodiments, R² is C₁₋₆ alkyl. In some embodiments, R² is C₄₋₁₀ cycloalkyl.

In some embodiments, R³ is H.

In some embodiments, R³ is NO₂.

In some embodiments, R³ is C₁₋₆ alkyl. In some embodiments, R³ is C₁₋₆ alkyl substituted with hydroxy. In some embodiments, R³ is ethyl substituted with hydroxy.

In some embodiments, R³ is C₄₋₁₀ cycloalkyl.

In some embodiments, R³ is C₁₋₆ alkyl(hetCyc¹). In some embodiments, C₁₋₆ alkyl is methyl, ethyl, or propyl. In some embodiments, C₁₋₆ alkyl is substituted with hydroxy. In some embodiments, C₁₋₆ alkyl is ethyl substituted with hydroxy. In some embodiments, hetCyc¹ is a 6-membered ring having two nitrogen ring atoms. In some embodiments, hetCyc¹ is substituted with C₁₋₆ alkyl. In some embodiments, hetCyc¹ is substituted with oxo. In some embodiments, hetCyc¹ is substituted with C₁₋₆ alkyl and oxo. In some embodiments, hetCyc¹ is piperazin-2-one optionally substituted with C₁₋₆ alkyl. In some embodiments, hetCyc¹ is 1-ethylpiperazine-2-one.

In some embodiments, R³ is C(═O)R⁹. In some embodiments, R⁹ is C₁₋₆ alkyl. In some embodiments, C₁₋₆ alkyl is methyl, ethyl, or propyl. In some embodiments, C₁₋₆ alkyl is substituted with halogen. In some embodiments, C₁₋₆ alkyl is methyl substituted with chlorine. In some embodiments, R⁹ is C₄₋₁₀ cycloalkyl. In some embodiments, R⁹ is C₁₋₆ alkyl(hetCyc¹). In some embodiments, C₁₋₆ alkyl is methyl, ethyl, or propyl. In some embodiments, hetCyc¹ is a 6-membered ring having two nitrogen ring atoms. In some embodiments, hetCyc¹ is piperazine. In some embodiments, hetCyc¹ is a 6-membered ring having one nitrogen ring atom and one oxygen ring atom. In some embodiments, hetCyc¹ is morpholine. In some embodiments, hetCyc¹ is substituted with one or two C₁₋₆ alkyl.

In some embodiments, R³ is SO₂(hetCyc¹). In some embodiments, hetCyc¹ is a 6-membered ring having two nitrogen atoms. In some embodiments, hetCyc¹ is a 6-membered ring having two nitrogen atoms substituted with one or two C₁₋₆ alkyl. In some embodiments, hetCyc¹ is 1-methylpiperazine. In some embodiments, hetCyc¹ is a 6-membered ring having two nitrogen atoms substituted with C₁₋₆ hydroxyalkyl. In some embodiments, hetCyc¹ is a 6-membered ring having two nitrogen atoms substituted with C₁₋₆ alkyl(aryl).

In some embodiments, R³ is SO₂NR¹⁰R¹¹. In some embodiments, R¹⁰ is H. In some embodiments, R¹⁰ is C₁₋₆ alkyl. In some embodiments, R¹⁰ is methyl, ethyl, or propyl. In some embodiments, R¹¹ is C₁₋₆ alkyl(NR′R″). In some embodiments, R′ is C₁₋₆ alkyl. In some embodiments, R′ is methyl, ethyl, or propyl. In some embodiments, R¹¹ is C₁₋₆ alkyl. In some embodiments, R″ is methyl, ethyl, or propyl. In some embodiments, R¹¹ is C₁₋₆ alkyl(hetCyc¹). In some embodiments, C₁₋₆ alkyl is methyl, ethyl, or propyl. In some embodiments, hetCyc¹ is a 5-membered ring having one nitrogen atom optionally substituted with C₁₋₆ alkyl. In some embodiments, hetCyc¹ is 1-methylpyrrolidine. In some embodiments, R¹¹ is (C₁₋₆ alkyl)C(═O)NR′(C₁₋₆ alkyl)C(═O)NR′R″. In some embodiments, each C₁₋₆ alkyl is independently selected from methyl, ethyl, and propyl. In some embodiments, each R′ is H. In some embodiments, each R′ is C₁₋₆ alkyl. In some embodiments, each R′ is independently selected from H and C₁₋₆ alkyl. In some embodiments, each C₁₋₆ alkyl is independently methyl, ethyl, or propyl.

In some embodiments, R³ is selected from

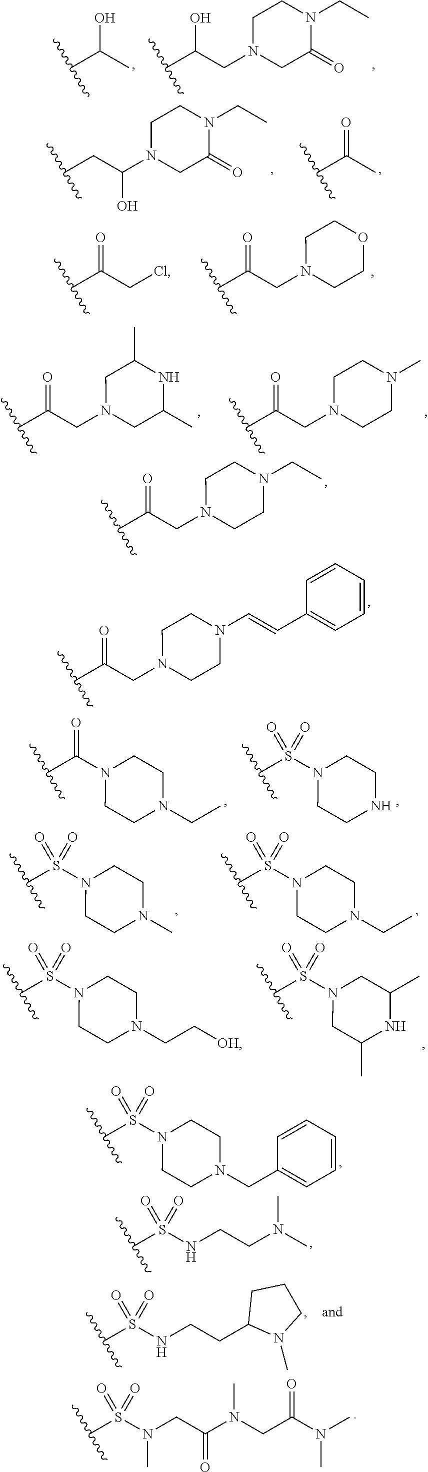

In some embodiments, R⁴ is H. In some embodiments, R⁴ is C₁₋₆ alkyl. In some embodiments, R⁴ is C₄₋₁₀ cycloalkyl. In some embodiments, R⁴ is C(═O)(C₁₋₆ alkyl). In some embodiments, R⁴ is C(═O)CH₃.

In some embodiments, provided is a compound of Formula I, or a pharmaceutically acceptable salt thereof, wherein:

Z is O;

W is N or CH;

X¹ and X² are each independently selected from N and NR⁶;

X³ is CR⁸;

X⁴ is C;

R¹ is H, C₁₋₆ alkyl, or -L-O—(C₁₋₆ alkyl);

R² is H;

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H;

R⁶ is H, C₁₋₆ alkyl, (C₁₋₆ alkyl)-O—(C₁₋₆ alkyl), aryl, C₁₋₆ alkyl(hetAr¹), or hetCyc¹;

R⁸ is C₁₋₆ alkyl;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1 nitrogen ring atom which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N and O which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

the dashed lines can be single or double bonds.

In some embodiments, the compound of Formula I is a compound of Formula Ia:

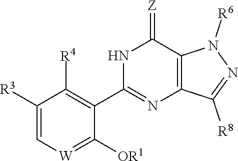

or a pharmaceutically acceptable salt thereof, wherein:

Z is O or S;

W is N or CR⁵;

R¹ is H, C₁₋₆ alkyl, or -L-O—(C₁₋₆ alkyl);

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, or C(═O)(C₁₋₆ alkyl);

R⁵ is H or C₁₋₆ alkyl;

R⁶ is H, C₁₋₆ alkyl, -L-O—(C₁₋₆ alkyl), -L-aryl, -L-hetAr¹, or -L-hetCyc¹;

R⁸ is H or C₁₋₆ alkyl;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, the compound of Formula I is a compound of Formula Ia, or a pharmaceutically acceptable salt thereof, wherein:

Z is O or S;

W is N or CH;

R¹ is C₁₋₆ alkyl;

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H;

R⁶ is C₁₋₆ alkyl;

R⁸ is C₁₋₆ alkyl;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl; and

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

In some embodiments, the compound of Formula I is a compound of Formula Ib:

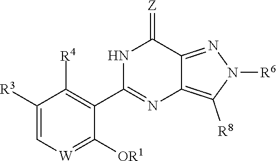

or a pharmaceutically acceptable salt thereof, wherein:

Z is O or S;

W is N or CR⁵;

R¹ is H, C₁₋₆ alkyl, or -L-O—(C₁₋₆ alkyl);

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, or C(═O)(C₁₋₆ alkyl);

R⁵ is H or C₁₋₆ alkyl;

R⁶ is H, C₁₋₆ alkyl, -L-O—(C₁₋₆ alkyl), -L-aryl, -L-hetAr¹, or -L-hetCyc¹;

R⁸ is H or C₁₋₆ alkyl;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, the compound of Formula I is a compound of Formula Ib, or a pharmaceutically acceptable salt thereof, wherein:

Z is O or S;

W is N or CH;

R¹ is C₁₋₆ alkyl or —(C₁₋₆ alkyl)-O—(C₁₋₆ alkyl);

R³ is H, C(═O)C₁₋₆ alkyl, or SO₂(hetCyc¹);

R⁴ is H or C(═O)(C₁₋₆ alkyl);

R⁶ is H, C₁₋₆ alkyl, -L-O—(C₁₋₆ alkyl), -L-aryl, -L-hetAr¹, or -L-hetCyc¹;

R⁸ is C₁₋₆ alkyl;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, the compound of Formula I is a compound of Formula Ic:

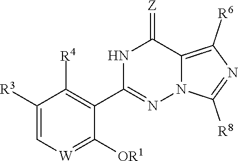

or a pharmaceutically acceptable salt thereof, wherein:

Z is O or S;

W is N or CR⁵;

R¹ is H, C₁₋₆ alkyl, or -L-O—(C₁₋₆ alkyl);

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, or C(═O)(C₁₋₆ alkyl);

R⁵ is H or C₁₋₆ alkyl;

R⁶ is H, C₁₋₆ alkyl, -L-O—(C₁₋₆ alkyl), -L-aryl, -L-hetAr¹, or -L-hetCyc¹;

R⁸ is H or C₁₋₆ alkyl;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, the compound of Formula I is a compound of Formula Ic, or a pharmaceutically acceptable salt thereof, wherein:

Z is O;

W is CH;

R¹ is C₁₋₆ alkyl;

R³ is SO₂(hetCyc¹) or SO₂NR¹⁰R¹¹;

R⁴ is H;

R⁶ is C₁₋₆ alkyl;

R⁸ is C₁₋₆ alkyl;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl; and

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

In some embodiments, the compound of Formula I is a compound of Formula Id:

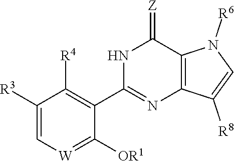

or a pharmaceutically acceptable salt thereof, wherein:

Z is O or S;

W is N or CR⁵;

R¹ is H, C₁₋₆ alkyl, or -L-O—(C₁₋₆ alkyl);

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, or C(═O)(C₁₋₆ alkyl);

R⁵ is H or C₁₋₆ alkyl;

R⁶ is H, C₁₋₆ alkyl, -L-O—(C₁₋₆ alkyl), -L-aryl, -L-hetAr¹, or -L-hetCyc¹;

R⁸ is H or C₁₋₆ alkyl;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, the compound of Formula I is a compound of Formula Id, or a pharmaceutically acceptable salt thereof, wherein:

Z is O;

W is CH;

R¹ is C₁₋₆ alkyl;

R³ is SO₂(hetCyc¹);

R⁴ is H;

R⁶ is C₁₋₆ alkyl;

R⁸ is C₁₋₆ alkyl; and

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

In some embodiments, the compound of Formula I is a compound of Formula Ie:

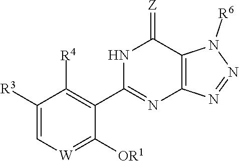

or a pharmaceutically acceptable salt thereof, wherein:

Z is O or S;

W is N or CR⁵;

R¹ is H, C₁₋₆ alkyl, or -L-O—(C₁₋₆ alkyl);

R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;

R⁴ is H, C₁₋₆ alkyl, or C(═O)(C₁₋₆ alkyl);

R⁵ is H or C₁₋₆ alkyl;

R⁶ is H, C₁₋₆ alkyl, -L-O—(C₁₋₆ alkyl), -L-aryl, -L-hetAr¹, or -L-hetCyc¹;

R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;

R¹⁰ is H or C₁₋₆ alkyl;

R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

L is absent or C₁₋₆ alkyl.

In some embodiments, the compound of Formula I is a compound of Formula Ie, or a pharmaceutically acceptable salt thereof, wherein:

Z is O;

W is CH;

R¹ is C₁₋₆ alkyl;

R³ is H;

R⁴ is H; and

R⁶ is H.

In some embodiments, the compound of Formula I is any one of the compounds listed in Table 1, or an enantiomer, tautomer, or pharmaceutically acceptable salt thereof.

TABLE 1

| # | Structure |
|---|---|
| 1 | 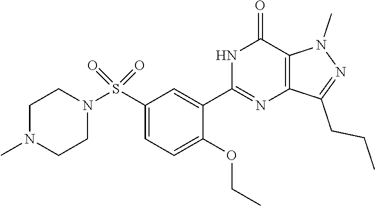 [SMILES: CCCc1nn(C)c2c(=O)[nH]c(-c3cc(S(=O)(=O)N4CCN(C)CC4)ccc3OCC)nc12] |
| 2 | 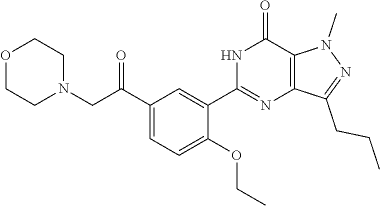 [SMILES: CCCc1nn(C)c2c(=O)[nH]c(-c3cc(C(=O)CN4CCOCC4)ccc3OCC)nc12] |
| 3 | 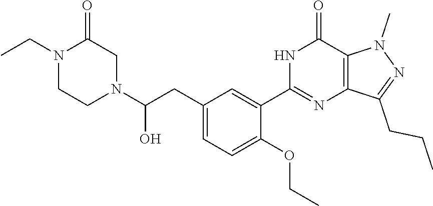 [SMILES: CCCc1nn(C)c2c(=O)[nH]c(-c3cc(CC(O)N4CCN(CC)C(=O)C4)ccc3OCC)nc12] |
| 4 | 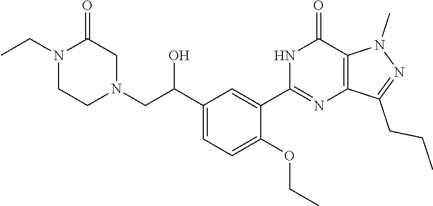 [SMILES: CCCc1nn(C)c2c(=O)[nH]c(-c3cc(C(O)CN4CCN(CC)C(=O)C4)ccc3OCC)nc12] |
| 5 | 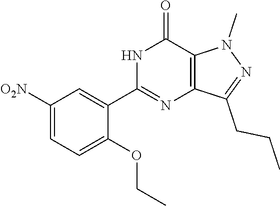 [SMILES: CCCc1nn(C)c2c(=O)[nH]c(-c3cc([N+](=O)[O-])ccc3OCC)nc12] |
| 6 | 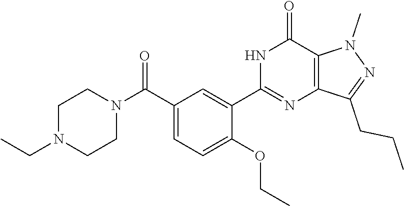 [SMILES: CCCc1nn(C)c2c(=O)[nH]c(-c3cc(C(=O)N4CCN(CC)CC4)ccc3OCC)nc12] |
| 7 | 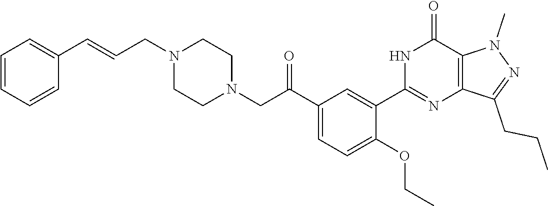 [SMILES: CCCc1nn(C)c2c(=O)[nH]c(-c3cc(C(=O)CN4CCN(C/C=C/c5ccccc5)CC4)ccc3OCC)nc12] |
| 8 | 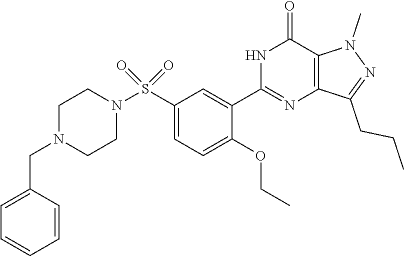 [SMILES: CCCc1nn(C)c2c(=O)[nH]c(-c3cc(S(=O)(=O)N4CCN(Cc5ccccc5)CC4)ccc3OCC)nc12] |
| 9 | 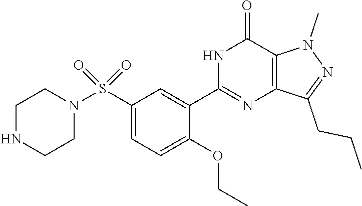 [SMILES: CCCc1nn(C)c2c(=O)[nH]c(-c3cc(S(=O)(=O)N4CCNCC4)ccc3OCC)nc12] |
| 10 | 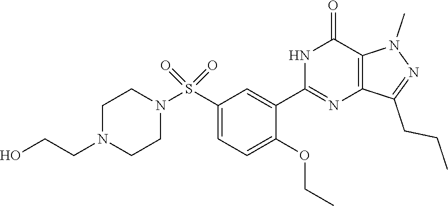 [SMILES: CCCc1nn(C)c2c(=O)[nH]c(-c3cc(S(=O)(=O)N4CCN(CCO)CC4)ccc3OCC)nc12] |
| 11 | 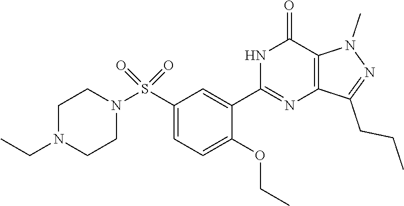 [SMILES: CCCc1nn(C)c2c(=O)[nH]c(-c3cc(S(=O)(=O)N4CCN(CC)CC4)ccc3OCC)nc12] |
| 12 | 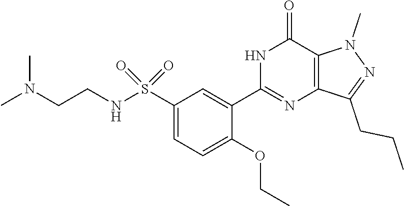 [SMILES: CCCc1nn(C)c2c(=O)[nH]c(-c3cc(S(=O)(=O)NCCN(C)C)ccc3OCC)nc12] |
| 13 | 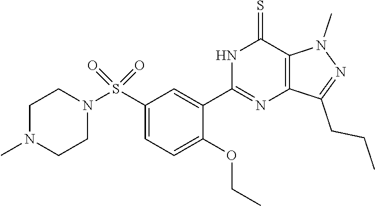 [SMILES: CCCc1nn(C)c2c(=S)[nH]c(-c3cc(S(=O)(=O)N4CCN(C)CC4)ccc3OCC)nc12] |
| 14 | 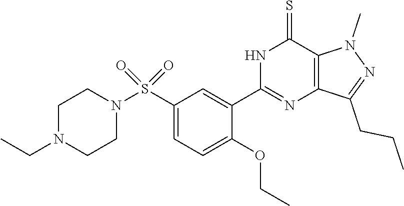 [SMILES: CCCc1nn(C)c2c(=S)[nH]c(-c3cc(S(=O)(=O)N4CCN(CC)CC4)ccc3OCC)nc12] |
| 15 | 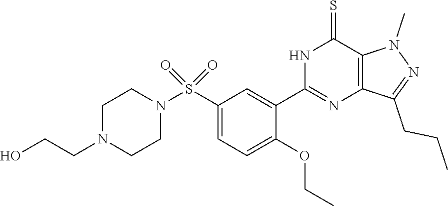 [SMILES: CCCc1nn(C)c2c(=S)[nH]c(-c3cc(S(=O)(=O)N4CCN(CCO)CC4)ccc3OCC)nc12] |
| 16 | 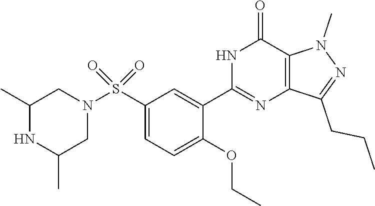 [SMILES: CCCc1nn(C)c2c(=O)[nH]c(-c3cc(S(=O)(=O)N4CC(C)NC(C)C4)ccc3OCC)nc12] |
| 17 | 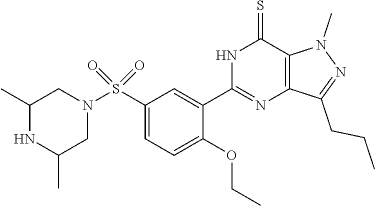 [SMILES: CCCc1nn(C)c2c(=S)[nH]c(-c3cc(S(=O)(=O)N4CC(C)NC(C)C4)ccc3OCC)nc12] |
| 18 | 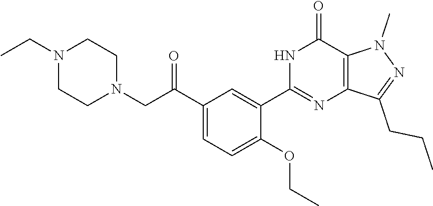 [SMILES: CCCc1nn(C)c2c(=O)[nH]c(-c3cc(C(=O)CN4CCN(CC)CC4)ccc3OCC)nc12] |
| 19 | 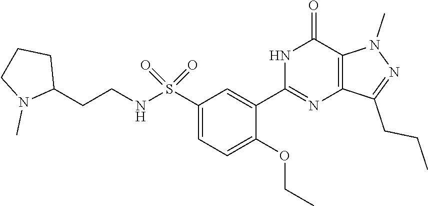 [SMILES: CCCc1nn(C)c2c(=O)[nH]c(-c3cc(S(=O)(=O)NCCC4CCCN4C)ccc3OCC)nc12] |
| 20 | 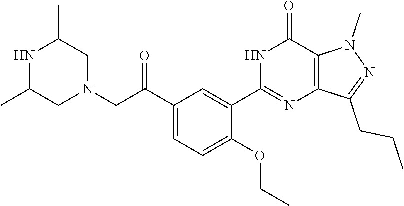 [SMILES: CCCc1nn(C)c2c(=O)[nH]c(-c3cc(C(=O)CN4CC(C)NC(C)C4)ccc3OCC)nc12] |
| 21 | 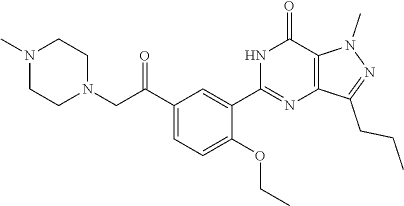 [SMILES: CCCc1nn(C)c2c(=O)[nH]c(-c3cc(C(=O)CN4CCN(C)CC4)ccc3OCC)nc12] |
| 22 | 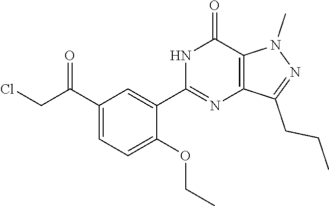 [SMILES: CCCc1nn(C)c2c(=O)[nH]c(-c3cc(C(=O)CCl)ccc3OCC)nc12] |
| 23 | 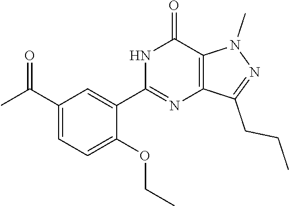 [SMILES: CCCc1nn(C)c2c(=O)[nH]c(-c3cc(C(C)=O)ccc3OCC)nc12] |
| 24 | 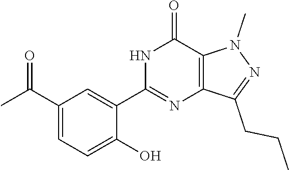 [SMILES: CCCc1nn(C)c2c(=O)[nH]c(-c3cc(C(C)=O)ccc3O)nc12] |
| 25 | 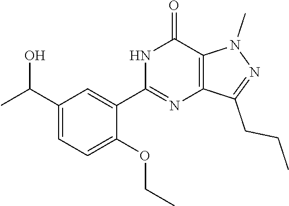 [SMILES: CCCc1nn(C)c2c(=O)[nH]c(-c3cc(C(C)O)ccc3OCC)nc12] |
| 26 | 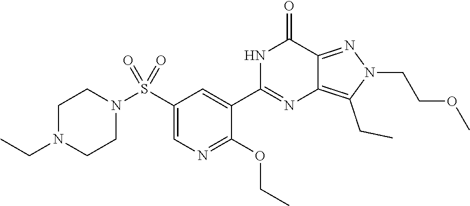 [SMILES: CCOc1ncc(S(=O)(=O)N2CCN(CC)CC2)cc1-c1nc2c(CC)n(CCOC)nc2c(=O)[nH]1] |
| 27 | 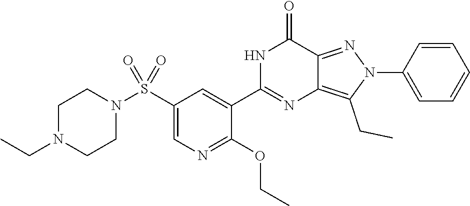 [SMILES: CCOc1ncc(S(=O)(=O)N2CCN(CC)CC2)cc1-c1nc2c(CC)n(-c3ccccc3)nc2c(=O)[nH]1] |
| 28 | 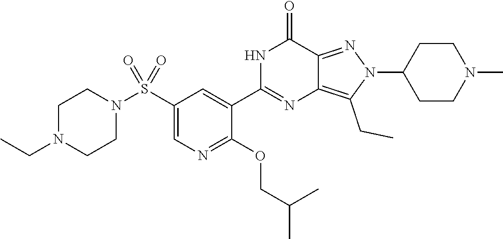 [SMILES: CCc1c2nc(-c3cc(S(=O)(=O)N4CCN(CC)CC4)cnc3OCC(C)C)[nH]c(=O)c2nn1C1CCN(C)CC1] |
| 29 | 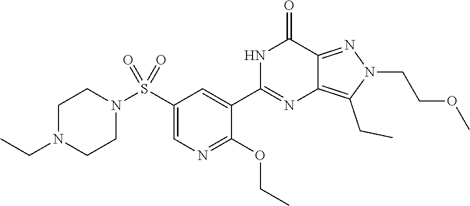 [SMILES: CCOc1ncc(S(=O)(=O)N2CCN(CC)CC2)cc1-c1nc2c(CC)n(CCOC)nc2c(=O)[nH]1] |
| 30 | 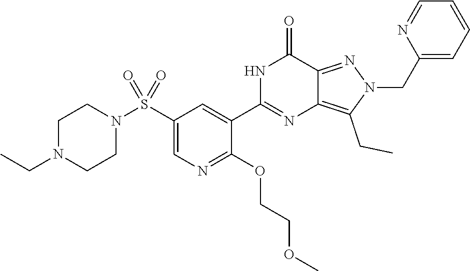 [SMILES: CCc1c2nc(-c3cc(S(=O)(=O)N4CCN(CC)CC4)cnc3OCCOC)[nH]c(=O)c2nn1Cc1ccccn1] |
| 31 | 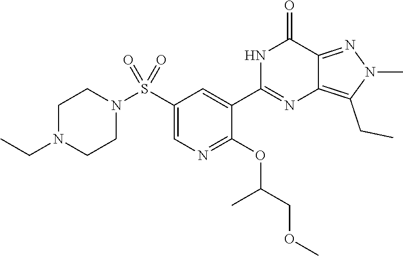 [SMILES: CCc1c2nc(-c3cc(S(=O)(=O)N4CCN(CC)CC4)cnc3OC(C)COC)[nH]c(=O)c2nn1C] |
| 32 | 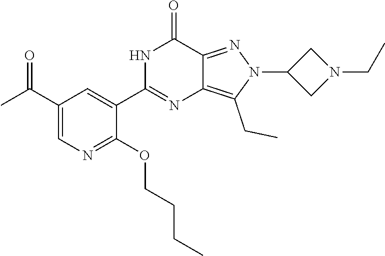 [SMILES: CCCCOc1ncc(C(C)=O)cc1-c1nc2c(CC)n(C3CN(CC)C3)nc2c(=O)[nH]1] |
| 33 | 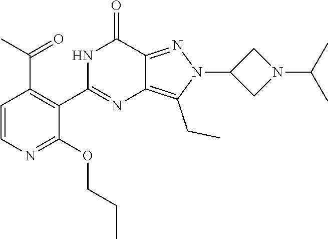 [SMILES: CCCOc1nccc(C(C)=O)c1-c1nc2c(CC)n(C3CN(C(C)C)C3)nc2c(=O)[nH]1] |
| 34 | 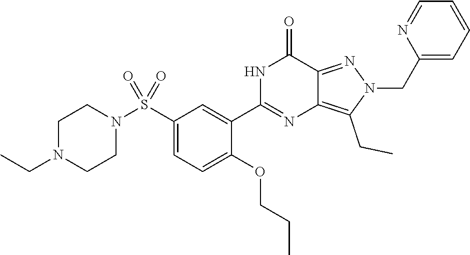 [SMILES: CCCOc1ccc(S(=O)(=O)N2CCN(CC)CC2)cc1-c1nc2c(CC)n(Cc3ccccn3)nc2c(=O)[nH]1] |
| 35 | 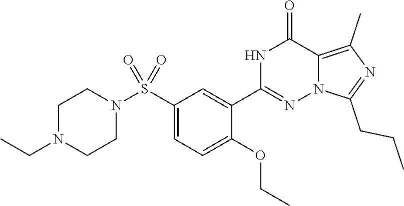 [SMILES: CCCc1nc(C)c2c(=O)[nH]c(-c3cc(S(=O)(=O)N4CCN(CC)CC4)ccc3OCC)nn12] |
| 36 | 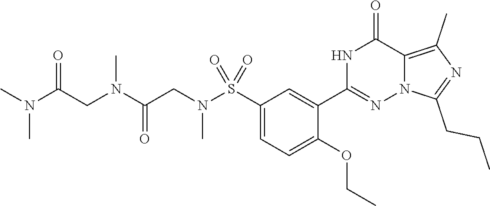 [SMILES: CCCc1nc(C)c2c(=O)[nH]c(-c3cc(S(=O)(=O)N(C)CC(=O)N(C)CC(=O)N(C)C)ccc3OCC)nn12] |
| 37 | 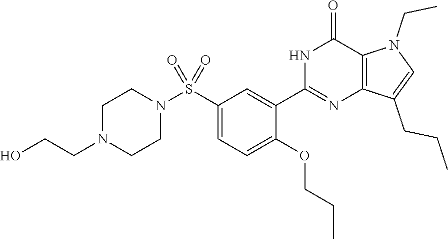 [SMILES: CCCOc1ccc(S(=O)(=O)N2CCN(CCO)CC2)cc1-c1nc2c(CCC)cn(CC)c2c(=O)[nH]1] |
| 38 | 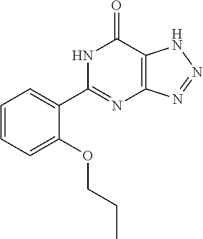 [SMILES: CCCOc1ccccc1-c1nc2nn[nH]c2c(=O)[nH]1] |

In some embodiments, the PDE5 inhibitor is sildenafil compound 1):

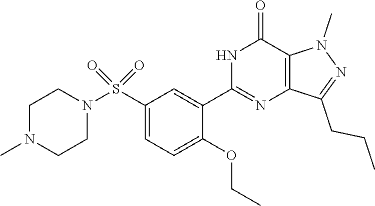 [SMILES: CCCc1nn(C)c2c(=O)[nH]c(-c3cc(S(=O)(=O)N4CCN(C)CC4)ccc3OCC)nc12]

or a pharmaceutically acceptable salt thereof.

In some embodiments, the PDE5 inhibitor is udenafil (compound 19):

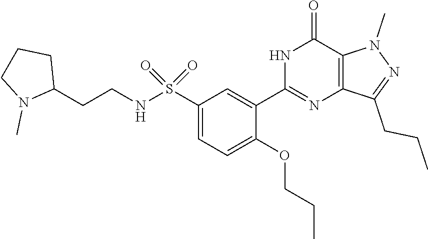 [SMILES: CCCOc1ccc(S(=O)(=O)NCCC2CCCN2C)cc1-c1nc2c(CCC)nn(C)c2c(=O)[nH]1]

or a pharmaceutically acceptable salt thereof.

In some embodiments, the PDE5 inhibitor is vardenafil (compound 35):

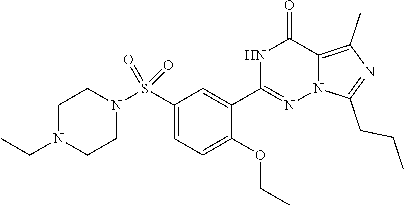 [SMILES: CCCc1nc(C)c2c(=O)[nH]c(-c3cc(S(=O)(=O)N4CCN(CC)CC4)ccc3OCC)nn12]

or a pharmaceutically acceptable salt thereof.

In some embodiments of the method, the PDE5 inhibitor is a compound of Formula II:

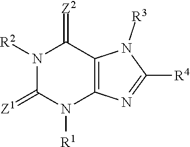

and pharmaceutically acceptable salts thereof, wherein:

Z¹ is O or S;

Z² is O or S;

R¹ is H, C₁₋₆ alkyl, C₄₋₁₀ cycloalkyl, or C₁₋₆ hydroxyalkyl;

R² is H, C₁₋₆ alkyl, C₄₋₁₀ cycloalkyl, or C₁₋₆ hydroxyalkyl;

R³ is H, C₁₋₆ alkyl, C₄₋₁₀ cycloalkyl, C₁₋₆ alkyl(hetCyc¹), C₁₋₆ alkyl(hetAr¹), or C₁₋₆ alkyl(aryl), wherein any C₁₋₆ alkyl is optionally substituted with one or more hydroxy and halogen, and aryl is optionally substituted with one or more substituents independently selected from halogen, C₁₋₆ alkyl, amino, cyano, C₁₋₆ alkoxy, and hydroxy;

R⁴ is H, C₁₋₆ alkyl, C₄₋₁₀ cycloalkyl, or NR′R″, wherein R′ and R″ are each independently selected from H, C₁₋₆ alkyl, C₄₋₁₀ cycloalkyl, hetCyc¹, hetAr¹, aryl, C₁₋₆ alkyl(hetCyc¹), C₁₋₆ alkyl(hetAr¹), and C₁₋₆ alkyl(aryl), and wherein any C₁₋₆ alkyl or C₄₋₁₀ cycloalkyl is optionally substituted with one or more hydroxy and halogen, and aryl is optionally substituted with one or more substituents independently selected from halogen, C₁₋₆ alkyl, amino, cyano, C₁₋₆ alkoxy, and hydroxy;

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy; and

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

In some embodiments, Z¹ is O. In some embodiments, Z¹ is S.

In some embodiments, Z² is O. In some embodiments, Z² is S.

In some embodiments, Z¹ and Z² are each O.

In some embodiments, R¹ is H. In some embodiments, R¹ is C₁₋₆ alkyl. In some embodiments, R¹ is C₁₋₆ hydroxyalkyl. In some embodiments, R¹ is C₁₋₃ hydroxyalkyl. In some embodiments, R¹ is C₄₋₁₀ cycloalkyl.

In some embodiments, R² is H. In some embodiments, R² is C₁₋₆ alkyl. In some embodiments, R² is methyl, ethyl, or propyl. In some embodiments, R² is C₁₋₆ hydroxyalkyl. In some embodiments, R² is C₄₋₁₀ cycloalkyl.

In some embodiments, R³ is H. In some embodiments, R³ is C₁₋₆ alkyl. In some embodiments, R³ is C₄₋₁₀ cycloalkyl. In some embodiments, R³ is C₁₋₆ alkyl(hetCyc¹). In some embodiments, R³ is C₁₋₆ alkyl(hetAr¹). In some embodiments, R³ is C₁₋₆ alkyl(aryl). In some embodiments, R³ is C₁₋₆ alkyl(aryl) substituted with one or two substituents independently selected from halogen and C₁₋₆ alkoxy.

In some embodiments, R⁴ is H. In some embodiments, R⁴ is C₁₋₆ alkyl. In some embodiments, R⁴ is C₄₋₁₀ cycloalkyl. In some embodiments, R⁴ is NR′R″, wherein R′ and R″ are each independently selected from H, C₁₋₆ alkyl, C₄₋₁₀ cycloalkyl, hetCyc¹, hetAr¹, aryl, C₁₋₆ alkyl(hetCyc¹), C₁₋₆ alkyl(hetAr¹), and C₁₋₆ alkyl(aryl). In some embodiments, R′ is H. In some embodiments, R″ is C₄₋₁₀ cycloalkyl. In some embodiments, R″ is cyclopentane. In some embodiments, R″ is cyclopentane substituted with hydroxy.

In some embodiments, the compound of Formula II is the compound:

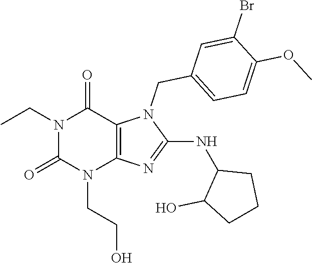 [SMILES: CCn1c(=O)c2c(nc(NC3CCCC3O)n2Cc2ccc(OC)c(Br)c2)n(CCO)c1=O]

or a pharmaceutically acceptable salt thereof.

In some embodiments of the method, the PDE5 inhibitor is a compound of Formula III:

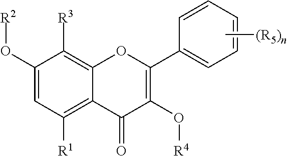

and pharmaceutically acceptable salts thereof, wherein:

R¹ is H, hydroxy, C₁₋₆ alkyl, C₄₋₁₀ cycloalkyl, or C₁₋₆ hydroxyalkyl;

R² is H, C₁₋₆ alkyl, C4-10 cycloalkyl, or hetCyc¹;

R³ is H, C₁₋₆ alkyl, C4-10 cycloalkyl, or C₂₋₁₀ alkenyl;

R⁴ is H, C₁₋₆ alkyl, C4-10 cycloalkyl, or hetCyc¹;

R⁵ is H, C₁₋₆ alkyl, C4-10 cycloalkyl, or C₁₋₆ alkoxy;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

n is 0 to 5.

In some embodiments, R¹ is H. In some embodiments, R¹ is hydroxy. In some embodiments, R¹ is C₁₋₆ alkyl. In some embodiments, R¹ is C₄₋₁₀ cycloalkyl. In some embodiments, R¹ is C₁₋₆ hydroxyalkyl.

In some embodiments, R² is H. In some embodiments, R² is C₁₋₆ alkyl. In some embodiments, R² is C₄₋₁₀ cycloalkyl. In some embodiments, R² is or hetCyc¹. In some embodiments, hetCyc¹ is tetrahydropyran. In some embodiments, hetCyc¹ is tetrahydropyran substituted with one to four substituents independently selected from hydroxy and C₁₋₃ hydroxyalkyl.

In some embodiments, R³ is H. In some embodiments, R³ is C₁₋₆ alkyl. In some embodiments, R³ is C₄₋₁₀ cycloalkyl. In some embodiments, R³ is C₂₋₁₀ alkenyl. In some embodiments, R³ is C₅ alkenyl.

In some embodiments, R⁴ is H. In some embodiments, R⁴ is C₁₋₆ alkyl. In some embodiments, R⁴ is C₄₋₁₀ cycloalkyl. In some embodiments, R⁴ is hetCyc¹. In some embodiments, hetCyc¹ is tetrahydropyran. In some embodiments, hetCyc¹ is tetrahydropyran substituted with one to four substituents independently selected from hydroxy and C₁₋₃ alkyl.

In some embodiments, R⁵ is H. In some embodiments, R⁵ is C₁₋₆ alkyl. In some embodiments, R⁵ is C₄₋₁₀ cycloalkyl. In some embodiments, R⁵ is C₁₋₆ alkoxy. In some embodiments, R⁵ is methoxy. In some embodiments, n is 1.

In some embodiments, the compound of Formula III is the compound:

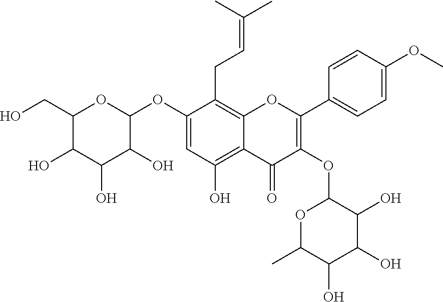 [SMILES: COc1ccc(-c2oc3c(CC=C(C)C)c(OC4OC(CO)C(O)C(O)C4O)cc(O)c3c(=O)c2OC2OC(C)C(O)C(O)C2O)cc1]

or a pharmaceutically acceptable salt thereof. In some embodiments of the method, the PDE5 inhibitor is a compound of Formula IV:

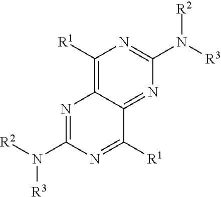

and pharmaceutically acceptable salts thereof, wherein:

R¹ is H, amino, C₁₋₆ alkyl, C₁₋₆ hydroxyalkyl, C₁₋₆ alkoxy, C₄₋₁₀ cycloalkyl, or hetCyc¹;

R² and R³ are each independently selected from H, C₁₋₆ alkyl, C₁₋₆ hydroxyalkyl, C₁₋₆ alkoxy, C₄₋₁₀ cycloalkyl, and hetCyc¹; and

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

In some embodiments, R¹ is H. In some embodiments, R¹ is amino. In some embodiments, R¹ is C₁₋₆ alkyl. In some embodiments, R¹ is C₁₋₆ hydroxyalkyl. In some embodiments, R¹ is C₁₋₆ alkoxy. In some embodiments, R¹ is C₄₋₁₀ cycloalkyl. In some embodiments, R¹ is hetCyc¹. In some embodiments, hetCyc¹ is a 6-membered ring having one nitrogen atom.

In some embodiments, R² is H. In some embodiments, R² is C₁₋₆ alkyl. In some embodiments, R² is C₁₋₆ hydroxyalkyl. In some embodiments, R² is C₁₋₃ hydroxyalkyl. In some embodiments, R² is C2 hydroxyalkyl. In some embodiments, R² is C₁₋₆ alkoxy. In some embodiments, R² is C₄₋₁₀ cycloalkyl. In some embodiments, R² is hetCyc¹.

In some embodiments, R³ is H. In some embodiments, R³ is C₁₋₆ alkyl. In some embodiments, R³ is C₁₋₆ hydroxyalkyl. In some embodiments, R³ is C₁₋₃ hydroxyalkyl. In some embodiments, R³ is C2 hydroxyalkyl. In some embodiments, R³ is C₁₋₆ alkoxy. In some embodiments, R³ is C₄₋₁₀ cycloalkyl. In some embodiments, R³ is hetCyc¹.

In some embodiments, R² and R³ are each C₁₋₃ hydroxyalkyl. In some embodiments, R² and R³ are each C2 hydroxyalkyl.

In some embodiments, the compound of Formula IV is dipyridamole, having the structure:

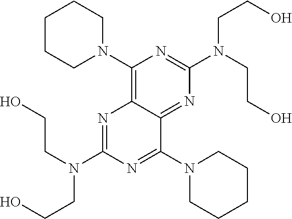 [SMILES: OCCN(CCO)c1nc(N2CCCCC2)c2nc(N(CCO)CCO)nc(N3CCCCC3)c2n1]

or a pharmaceutically acceptable salt thereof.

In some embodiments of the method, the PDE5 inhibitor is a compound of Formula V:

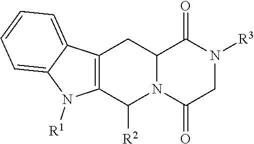

and pharmaceutically acceptable salts thereof, wherein:

R¹, R², and R³ are each independently selected from H, C₁₋₆ alkyl, C₁₋₆ hydroxyalkyl, C₁₋₆ alkoxy, C₄₋₁₀ cycloalkyl, and hetCyc¹; and

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

In some embodiments, R¹ is H. In some embodiments, R¹ is C₁₋₆ alkyl. In some embodiments, R¹ is C₁₋₆ hydroxyalkyl. In some embodiments, R¹ is C₁₋₆ alkoxy. In some embodiments, R¹ is C₄₋₁₀ cycloalkyl. In some embodiments, R¹ is hetCyc¹.

In some embodiments, R² is H. In some embodiments, R² is C₁₋₆ alkyl. In some embodiments, R² is C₁₋₆ hydroxyalkyl. In some embodiments, R² is C₁₋₆ alkoxy. In some embodiments, R² is C₄₋₁₀ cycloalkyl. In some embodiments, R² is hetCyc¹. In some embodiments, hetCyc¹ is a 6-10 membered bicyclic ring having one or two ring oxygen atoms. In some embodiments, hetCyc¹ is a 9-membered bicyclic ring having two ring oxygen atoms. In some embodiments, hetCyc¹ is 1,3-benzodioxole.

In some embodiments, R³ is H. In some embodiments, R³ is C₁₋₆ alkyl. In some embodiments, R³ is methyl, ethyl, or propyl. In some embodiments, R³ is C₁₋₆ hydroxyalkyl. In some embodiments, R³ is C₁₋₆ alkoxy. In some embodiments, R³ is C₄₋₁₀ cycloalkyl. In some embodiments, R³ is hetCyc¹.

In some embodiments, the compound of Formula V is tadalafil, having the structure:

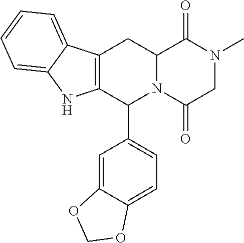 [SMILES: CN1CC(=O)N2C(Cc3c([nH]c4ccccc34)C2c2ccc3c(c2)OCO3)C1=O]

or a pharmaceutically acceptable salt thereof.

In some embodiments of the method, the PDE5 inhibitor is a compound of Formula VI:

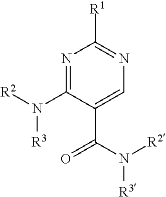

and pharmaceutically acceptable salts thereof, wherein:

R¹ is H, C₁₋₆ alkyl, C₁₋₆ hydroxyalkyl, C₁₋₆ alkoxy, C₄₋₁₀ cycloalkyl, or hetCyc¹;

R², R^{2′}, R³ and R^{3′} are each independently selected from H, C₁₋₆ alkyl, C₁₋₆ hydroxyalkyl, C₁₋₆ alkoxy, C₄₋₁₀ cycloalkyl, aryl, hetCyc¹, hetAr¹, C₁₋₆ alkyl(aryl), C₁₋₆ alkyl(hetCyc¹), and C₁₋₆ alkyl(hetAr¹), wherein aryl is optionally substituted with halogen, C₁₋₆ alkyl, C₁₋₆ alkoxy, amino, cyano, hydroxy, and C₁₋₆ hydroxyalkyl;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and

hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy.

In some embodiments, R¹ is H. In some embodiments, R¹ is C₁₋₆ alkyl. In some embodiments, R¹ is C₁₋₆ hydroxyalkyl. In some embodiments, R¹ is C₁₋₆ alkoxy. In some embodiments, R¹ is C₄₋₁₀ cycloalkyl. In some embodiments, R¹ is hetCyc¹. In some embodiments, hetCyc¹ is a 5-membered ring having one nitrogen atom. In some embodiments, hetCyc¹ is a 5-membered ring having one nitrogen atom substituted with C₁₋₃ hydroxyalkyl. In some embodiments, hetCyc¹ is pyrrolidine.

In some embodiments, R² is H. In some embodiments, R² is C₁₋₆ alkyl. In some embodiments, R² is C₁₋₆ hydroxyalkyl. In some embodiments, R² is C₁₋₆ alkoxy. In some embodiments, R² is C₄₋₁₀ cycloalkyl. In some embodiments, R² is aryl. In some embodiments, R² is hetCyc¹. In some embodiments, R² is hetAr¹. In some embodiments, R² is C₁₋₆ alkyl(aryl). In some embodiments, R² is C₁₋₆ alkyl(hetCyc¹). In some embodiments, R² is C₁₋₆ alkyl(hetAr¹).

In some embodiments, R^{2′} is H. In some embodiments, R^{2′} is C₁₋₆ alkyl. In some embodiments, R^{2′} is C₁₋₆ hydroxyalkyl. In some embodiments, R^{2′} is C₁₋₆ alkoxy. In some embodiments, R^{2′} is C₄₋₁₀ cycloalkyl. In some embodiments, R^{2′} is aryl. In some embodiments, R^{2′} is hetCyc¹. In some embodiments, R^{2′} is hetAr¹. In some embodiments, R^{2′} is C₁₋₆ alkyl(aryl). In some embodiments, R^{2′} is C₁₋₆ alkyl(hetCyc¹). In some embodiments, R^{2′} is C₁₋₆ alkyl(hetAr¹).

In some embodiments, R² and R^{2′} are each H.

In some embodiments, R³ is H. In some embodiments, R³ is C₁₋₆ alkyl. In some embodiments, R³ is C₁₋₆ hydroxyalkyl. In some embodiments, R³ is C₁₋₆ alkoxy. In some embodiments, R³ is C₄₋₁₀ cycloalkyl. In some embodiments, R³ is aryl. In some embodiments, R³ is hetCyc¹. In some embodiments, R³ is hetAr¹. In some embodiments, R³ is C₁₋₆ alkyl(aryl). In some embodiments, R³ is C₁₋₃ alkyl(aryl) substituted with one or two substituents selected from halogen and C₁₋₃ alkoxy. In some embodiments, R³ is C₁₋₆ alkyl(hetCyc¹). In some embodiments, R³ is C₁₋₆ alkyl(hetAr¹).

In some embodiments, R^{3′} is H. In some embodiments, R^{3′} is C₁₋₆ alkyl. In some embodiments, R^{3′} is C₁₋₆ hydroxyalkyl. In some embodiments, R^{3′} is C₁₋₆ alkoxy. In some embodiments, R^{3′} is C₄₋₁₀ cycloalkyl. In some embodiments, R^{3′} is aryl. In some embodiments, R^{3′} is hetCyc¹. In some embodiments, R^{3′} is hetAr¹. In some embodiments, R³ is C₁₋₆ alkyl(aryl). In some embodiments, R^{3′} is C₁₋₆ alkyl(hetCyc¹). In some embodiments, R^{3′} is C₁₋₆ alkyl(hetAr¹). In some embodiments, R^{3′} is C₁₋₃ alkyl(hetAr¹). In some embodiments, hetAr¹ is a 6-membered ring with two nitrogen ring atoms. In some embodiments, hetAr¹ is pyrimidine.

In some embodiments, the compound of Formula VI is avanafil, having the structure:

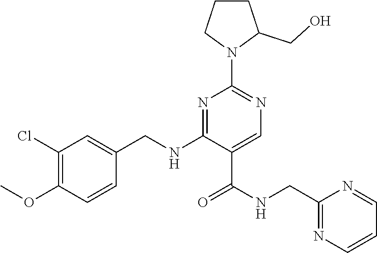 [SMILES: COc1ccc(CNc2nc(N3CCCC3CO)ncc2C(=O)NCc2ncccn2)cc1Cl]

or a pharmaceutically acceptable salt thereof.

In some embodiments of the method, the PDE5 inhibitor is a compound of Formula VII:

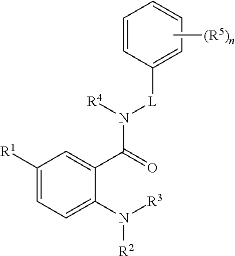

and pharmaceutically acceptable salts thereof, wherein:

R¹ is H, amino, nitro, C₁₋₆ alkyl, C₁₋₆ hydroxyalkyl, C₁₋₆ alkoxy, C₄₋₁₀ cycloalkyl, or hetCyc¹;

R², R³, and R⁴ are each independently selected from H, C₁₋₆ alkyl, C₁₋₆ hydroxyalkyl, C₁₋₆ alkoxy, C₄₋₁₀ cycloalkyl, and hetCyc¹;

R⁵ is H, amino, nitro, C₁₋₆ alkyl, C₁₋₆ hydroxyalkyl, C₁₋₆ alkoxy, C₄₋₁₀ cycloalkyl;

hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl);

L is —C₁₋₆ alkyl- or —C₁₋₆ alkoxy-; and

n is 0 to 5.

In some embodiments, R¹ is H. In some embodiments, R¹ is amino. In some embodiments, R¹ is nitro. In some embodiments, R¹ is C₁₋₆ alkyl. In some embodiments, R¹ is C₁₋₆ hydroxyalkyl. In some embodiments, R¹ is C₁₋₆ alkoxy. In some embodiments, R¹ is C4-cycloalkyl. In some embodiments, R¹ is hetCyc¹.

In some embodiments, R² is H. In some embodiments, R² is C₁₋₆ alkyl. In some embodiments, R² is C₁₋₆ hydroxyalkyl. In some embodiments, R² is C₁₋₆ alkoxy. In some embodiments, R² is C₄₋₁₀ cycloalkyl. In some embodiments, R² is hetCyc¹.

In some embodiments, R³ is H. In some embodiments, R³ is C₁₋₆ alkyl. In some embodiments, R³ is C₁₋₆ hydroxyalkyl. In some embodiments, R³ is C₁₋₃ hydroxyalkyl. In some embodiments, R³ is C3 hydroxyalkyl. In some embodiments, R³ is C₁₋₆ alkoxy. In some embodiments, R³ is C₄₋₁₀ cycloalkyl. In some embodiments, R³ is hetCyc¹.

In some embodiments, R⁴ is H. In some embodiments, R⁴ is C₁₋₆ alkyl. In some embodiments, R⁴ is C₁₋₆ hydroxyalkyl. In some embodiments, R⁴ is C₁₋₆ alkoxy. In some embodiments, R⁴ is C₄₋₁₀ cycloalkyl. In some embodiments, R⁴ is hetCyc¹.

In some embodiments, R⁵ is H. In some embodiments, R⁵ is amino. In some embodiments, R⁵ is nitro. In some embodiments, R⁵ is C₁₋₆ alkyl. In some embodiments, R⁵ is C₄₋₁₀ cycloalkyl. In some embodiments, R⁵ is C₁₋₆ hydroxyalkyl. In some embodiments, R⁵ is C₁₋₆ alkoxy. In some embodiments, R⁵ is C₁₋₃ alkoxy. In some embodiments, R⁵ is methoxy. In some embodiments, n is 0. In some embodiments, n is 1. In some embodiments, n is 2.

In some embodiments, L is —C₁₋₆ alkyl-. In some embodiments, L is —C₁₋₃ alkyl-. In some embodiments, L is —C₁ alkyl-. In some embodiments, L is —C₁₋₆ alkoxy-.

In some embodiments, the compound of Formula VII is the compound:

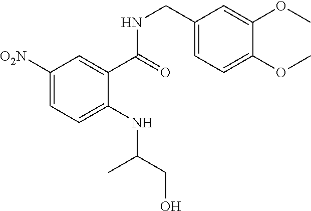 [SMILES: COc1ccc(CNC(=O)c2cc([N+](=O)[O-])ccc2NC(C)CO)cc1OC]

or a pharmaceutically acceptable salt thereof.

In some embodiments of the methods, the PDE5 inhibitor is any one of the compounds of Formulas I-VII, including any derivatives, enantiomers, active metabolites, or pharmaceutically acceptable salts thereof.

In some embodiments of the methods provided in the present disclosure, the PDE5 inhibitor is selected from sildenafil, tadalafil, vardenafil, udenafil, avanafil, and dipyridamole, or derivatives, enantiomers, active metabolites, or pharmaceutically acceptable salts thereof.

In some embodiments, the PDE5 inhibitor is selected from:

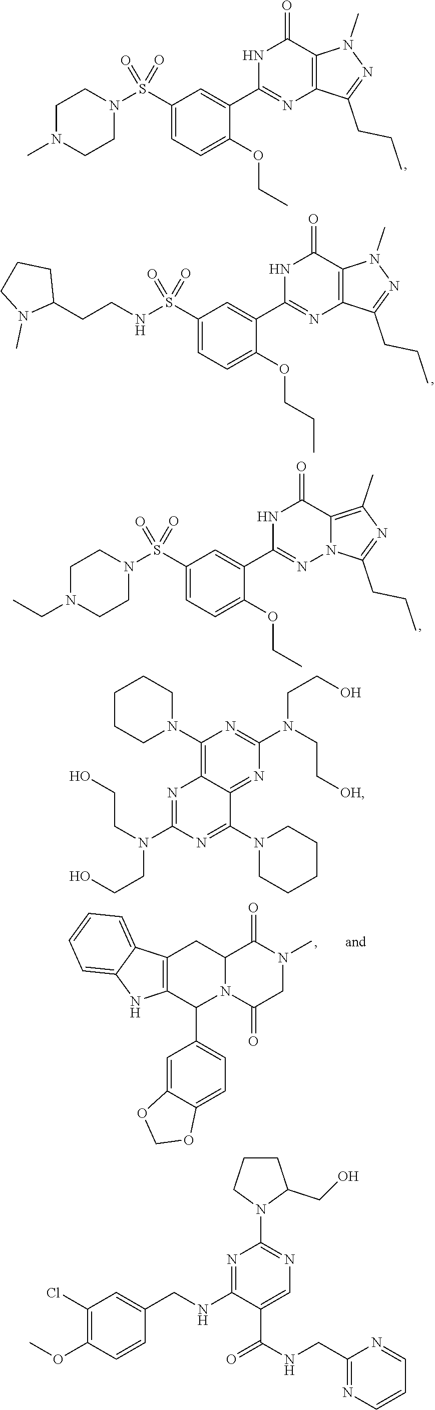

and pharmaceutically acceptable salts thereof.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As will be appreciated, the compounds provided herein, including salts thereof, can be prepared using known organic synthesis techniques and can be synthesized according to any of numerous possible synthetic routes. The compounds thus obtained can be further purified, for example, by flash column chromatography, high performance liquid chromatography, crystallization, or any known purification method.

Methods of making PDE5s are known in the art. For example, methods of making sildenafil are known in the art. See, for example, U.S. Pat. Nos. 6,066,735; 6,204,383; 6,592,850; 7,618,976; 8,071,765; 8,497,370; 8,808,739; 8,933,081; 9,186,321; 9,301,959; 9,662,334; 9,907,759; 9,968,609; 10,016,428; 10,092,651; 10,111,833; and 10,211,534. In addition, derivatives, enantiomers, active metabolites, or pharmaceutically acceptable salts of the compounds described herein are known in the art. Derivatives typically refer to precursors or compounds that derive from the original compound, while enantiomers refer to stereoisomers of a compound.

Methods of Use

Provided in the present disclosure are methods of reducing an individual's desire to smoke, the frequency of smoking, or both, that includes administering to the individual an amount of a PDE5 inhibitor that is effective to reduce the individual's desire to smoke, the frequency of smoking, or both. In some embodiments, the PDE5 inhibitor is a compound of Formulas I-VII or a pharmaceutically acceptable salt thereof as described herein. In some embodiments, the PDE5 inhibitor is sildenafil, a derivative of sildenafil, an enantiomer of sildenafil, an active metabolite of sildenafil, or a pharmaceutically acceptable salt of sildenafil.

As used herein, an effective amount of a PDE5 inhibitor refers to an amount that, following daily administration for a period of time (e.g., 1 week, 2 weeks, or 1 month), results in a reduction in the desire to smoke and/or frequency of smoking as reported by the individual or an actual reduction in the number or frequency of cigarettes smoked. In some embodiments, an effective amount for an average adult is a dose of about 1 mg/day to about 150 mg/day (e.g., about 1 mg/day to about 20 mg/day; about 1 mg/day to about 40 mg/day; about 1 mg/day to about 100 mg/day; about 5 mg/day to about 125 mg/day; about 5 mg/day to about 100 mg/day; about 10 mg/day to about 50 mg/day; about 10 mg/day to about 100 mg/day; about 25 mg/day to about 75 mg/day; about 50 mg/day to about 150 mg/day; about 75 mg/day to about 150 mg/day; about 100 mg/day to about 150 mg/day; about 30 mg/day to about 60 mg/day; about 5 mg/day to about 30 mg/day; about 10 mg/day to about 25 mg/day; or about 20 mg/day). In some embodiments, the PDE5 inhibitor is administered to the individual at a dose of about 1 mg/day to about 150 mg/day. In some embodiments, the amount of PDE5 inhibitor administered to the individual is the effective amount of the PDE5 inhibitor. In some embodiments, the PDE5 inhibitor is a compound of Formulas I-VII or a pharmaceutically acceptable salt thereof. In some embodiments, the PDE5 inhibitor is sildenafil, a derivative of sildenafil, an enantiomer of sildenafil, an active metabolite of sildenafil, or a pharmaceutically acceptable salt of sildenafil.

In some embodiments of the method, the PDE5 inhibitor is co-administered to the individual with another compound or medication. In some embodiments, the PDE5 inhibitor is co-administered with a nicotine replacement (e.g., in the form of gum or a transdermal patch), an antidepressant medication, such as bupropion (e.g., Wellbutrin®), a medication used to treat smoking addiction, such as varenicline (e.g., Chantix®), or an additional PDE5 inhibitor, such as any of the PDE5 inhibitors of Formulas I-VII described herein. In some embodiments, the additional PDE5 inhibitor is tadalafil (e.g., Cialis®). In some embodiments, the additional PDE5 inhibitor is vardenafil (Levitra®).

In some embodiments, the PDE5 inhibitor is administered with an effective amount of another compound or medication. Simply by way of example, typical doses of bupropion include about 100 mg/day-450 mg/day when being used to reduce an individual's desire to smoke and/or frequency of smoking, while typical doses of varenicline include about 0.5 mg/day to about 2 mg/day when being used to reduce an individual's desire to smoke and/or frequency of smoking.

In some embodiments, the method further includes applying one or more of motivational interviewing, incentive based programming, or other psychological technique(s) to the individual.

In some embodiments, following administration of a PDE5 inhibitor, individuals who experience a reduced desire to smoke and/or frequency of smoking exhibit improved lung diffusing capacity for carbon monoxide (DLCO), reduced parenchymal inflammation (or lung density), tissue re-perfusion, and combinations thereof. In some embodiments, the PDE5 inhibitor is sildenafil, a derivative of sildenafil, an enantiomer of sildenafil, an active metabolite of sildenafil, or a pharmaceutically acceptable salt of sildenafil.

Also provided in the present disclosure is a method of reducing an individual's desire to smoke and/or frequency of smoking, the method including administering an amount of a PDE5 inhibitor to the individual in an amount effective to reduce the individual's desire to smoke and/or frequency of smoking. In some embodiments, the PDE5 inhibitor is a compound of any of Formulas I-VII or a pharmaceutically acceptable salt thereof as described herein. In some embodiments, the PDE5 inhibitor is selected from sildenafil, tadalafil, vardenafil, udenafil, avanafil, and dipyridamole, and derivatives, enantiomers, active metabolites, and pharmaceutically acceptable salts thereof.

In some embodiments, the method further includes applying motivational interviewing, incentive based programming, or other psychological technique(s) to the individual.

Pharmaceutical Compositions and Formulations

Any of the PDE5 inhibitors described herein can be administered to an individual to reduce the individual's desire to smoke and/or frequency of smoking. Typically, the compounds described herein are formulated with a pharmaceutically acceptable carrier. Pharmaceutically acceptable carriers for delivering compounds are well known in the art. See, for example Remington: The Science and Practice of Pharmacy, University of the Sciences in Philadelphia, Ed., 21^{st} Edition, 2005, Lippincott Williams & Wilkins; and The Pharmacological Basis of Therapeutics, Goodman and Gilman, Eds., 12^{th} Ed., 2001, McGraw-Hill Co. The type of pharmaceutically acceptable carrier used in a particular formulation can depend on various factors, such as, for example, the physical and chemical properties of the compound, the route of administration, and the manufacturing procedure.

Pharmaceutically acceptable carriers are available in the art, and include those listed in various pharmacopoeias. See, for example, the U.S. Pharmacopeia (USP), Japanese Pharmacopoeia (JP), European Pharmacopoeia (EP), and British pharmacopeia (BP); the U.S. Food and Drug Administration (FDA) Center for Drug Evaluation and Research (CDER) publications (e.g., Inactive Ingredient Guide (1996)); and Ash and Ash, Eds. (2002) Handbook of Pharmaceutical Additives, Synapse Information Resources, Inc., Endicott, N.Y.

A pharmaceutical composition that includes a compound as described herein is typically formulated to be compatible with its intended route of administration. Suitable routes of administration include, for example, oral, topical, and parenteral administration. Routes for parenteral administration include intravenous, intramuscular, and subcutaneous administration, as well as intraperitoneal, intra-arterial, intra-articular, intracardiac, intracisternal, intradermal, intralesional, intraocular, intrapleural, intrathecal, intrauterine, and intraventricular administration.

In some embodiments, the PDE5 inhibitor is formulated for oral administration. For oral administration, a compound can be formulated in liquid or solid dosage forms, and also formulated as an instant release or controlled/sustained release formulations. In some embodiments, the PDE5 inhibitor is formulated for extended/delayed release. Suitable dosage forms for oral ingestion by an individual include tablets, pills, hard and soft shell capsules, liquids, gels, syrups, slurries, suspensions, and emulsions. For intravenous injection, for example, the composition may be formulated as an aqueous solution using physiologically compatible buffers, including, for example, phosphate, histidine, or citrate for adjustment of the formulation pH, and a tonicity agent, such as, for example, sodium chloride or dextrose.

The PDE5 inhibitor can be formulated for topical or transdermal administration. For example, the PDE5 inhibitor can be formulated as a dosage form that is a gel, an ointment, a cream, or a patch. In some embodiments, the PDE5 inhibitor is formulated as a patch. In some embodiments, the PDE5 inhibitor is formulated as a patch that also contains another compound or medication. In some embodiments, the other compound or medication is nicotine. In some embodiments, the dosage form contains one or more of an emulsification agent, a solubilization agent, a skin permeation enhance, and a detergent.

Solid oral dosage forms can be obtained using excipients, which can include fillers, disintegrants, binders (dry and wet), dissolution retardants, lubricants, glidants, anti-adherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatin, magnesium carbonate, magnesium/sodium lauryl sulfate, mannitol, polyethylene glycol, polyvinyl pyrrolidone, silicates, silicon dioxide, sodium benzoate, sorbitol, starches, stearic acid or a salt thereof, sugars (e.g., dextrose, sucrose, lactose), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. When a capsule is preferred over a tablet, the drug powder, suspension, or solution thereof can be delivered in a compatible hard or soft shell capsule. Any of the compounds described herein can be formulated in an extended release (or delayed release or time-delayed) formulation. Formulations for extended release are known in the art.

Articles of Manufacture

As described herein, an article of manufacture is provided that includes at least one dose of a PDE5 inhibitor, a derivative, an enantiomer, an active metabolite, or a pharmaceutically acceptable salt thereof. In some embodiments, the PDE5 inhibitor is a compound disclosed herein. In some embodiments, the PDE5 inhibitor is a compound of Formulas I-VII. In some embodiments, the PDE5 inhibitor is sildenafil (Viagra®), tadalafil (e.g., Cialis®), vardenafil (e.g., Levitra®), udenafil, avanafil, or dipyridamole. In some embodiments, the PDE5 inhibitor is sildenafil or a derivative, an enantiomer, an active metabolite, or a pharmaceutically acceptable salt thereof. As discussed herein, a dose of a PDE5 inhibitor is an amount that is effective to reduce an individual's desire to smoke and/or frequency of smoking and, thereby, increase the likelihood that an individual will stop smoking. In some embodiments, the dose of the PDE5 inhibitor is about 1 mg to about 150 mg. In some embodiments, the dose of the PDE5 inhibitor is about 1 mg to about 40 mg. In some embodiments, the PDE5 inhibitor is sildenafil.

In some embodiments, an article of manufacture further includes at least one dose of a nicotine replacement (e.g., in the form of gum or a transdermal patch), bupropion (e.g., Wellbutrin®) or varenicline (e.g., Chantix®), or derivatives, enantiomers, metabolites, or pharmaceutically acceptable salts thereof effective to reduce an individual's desire to smoke and/or frequency of smoking to increase the likelihood that an individual will stop smoking.

Articles of manufacture provided herein also can contain a package insert or package label having instructions thereon for using (e.g., ingesting) the compound(s). Articles of manufacture may additionally include further doses of the compound(s) contained within the article of manufacture.

Thus, provided in the present disclosure is an article of manufacture that includes at least one dose of a PDE5 inhibitor in an amount effective to reduce an individual's desire to smoke and/or frequency of smoking; and at least one dose of a nicotine replacement (e.g., in the form of gum or a transdermal patch), an antidepressant medication, a medication used to treat smoking addiction, or an additional PDE5 inhibitor in an amount effective to reduce an individual's desire to smoke and/or frequency of smoking. In some embodiments, the PDE5 inhibitor is sildenafil, a derivative of sildenafil, an enantiomer of sildenafil, an active metabolite of sildenafil, or a pharmaceutically acceptable salt of sildenafil. In some embodiments, the antidepressant medication is bupropion (e.g., Wellbutrin®). In some embodiments, the medication used to treat smoking addiction is varenicline (e.g., Chantix®). In some embodiments, the additional PDE5 inhibitor is selected from tadalafil (e.g., Cialis®) and vardenafil (e.g., Levitra®).

In some embodiments, the at least one dose of the PDE5 inhibitor is about 1 mg to about 150 mg. In some embodiments, the at least one dose of the PDE5 inhibitor is about 1 mg to about 40 mg. In some embodiments, the PDE5 inhibitor is sildenafil, a derivative of sildenafil, an enantiomer of sildenafil, an active metabolite of sildenafil, or a pharmaceutically acceptable salt of sildenafil.

In some embodiments, the PDE5 inhibitor is formulated for oral administration. In some embodiments, the PDE5 inhibitor is formulated for extended/delayed release. In some embodiments, the PDE5 inhibitor is sildenafil, a derivative of sildenafil, an enantiomer of sildenafil, an active metabolite of sildenafil, or a pharmaceutically acceptable salt of sildenafil.

Also provided in the present disclosure is an article of manufacture that includes at least one dose of sildenafil, a derivative of sildenafil, an enantiomer of sildenafil, an active metabolite of sildenafil, or a pharmaceutically acceptable salt of sildenafil, in an amount effective to reduce an individual's desire to smoke and/or frequency of smoking; and at least one dose of a nicotine replacement (e.g., in the form of gum or a transdermal patch), an antidepressant medication, a medication used to treat smoking addiction, or an additional PDE5 inhibitor in an amount effective to reduce an individual's desire to smoke and/or frequency of smoking. In some embodiments, the antidepressant medication is bupropion (e.g., Wellbutrin®). In some embodiments, the medication used to treat smoking addiction is varenicline (e.g., Chantix®). In some embodiments, the additional PDE5 inhibitor is selected from tadalafil (e.g., Cialis®) and vardenafil (e.g., Levitra®).

In some embodiments, the at least one dose of the sildenafil, derivative of sildenafil, enantiomer of sildenafil, active metabolite of sildenafil, or pharmaceutically acceptable salt of sildenafil is about 1 mg to about 150 mg. In some embodiments, the at least one dose of the sildenafil, derivative of sildenafil, enantiomer of sildenafil, active metabolite of sildenafil, or pharmaceutically acceptable salt of sildenafil is about 1 mg to about 40 mg.

In some embodiments, the sildenafil, derivative of sildenafil, enantiomer of sildenafil, active metabolite of sildenafil, or pharmaceutically acceptable salt of sildenafil is formulated for oral administration. In some embodiments, the sildenafil, derivative of sildenafil, enantiomer of sildenafil, active metabolite of sildenafil, or pharmaceutically acceptable salt of sildenafil is formulated for extended/delayed release.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Experimental Design and Preliminary Results

Smokers interested in stopping smoking were imaged using a dual energy computerized tomography (DECT) protocol to determine baseline lung density and perfusion, then randomized to receive either 20 mg of sildenafil or placebo each day. Subjects were then followed each month for three months (i.e., 30, 60 and 90 days) to asses changes in lung density via DECT and alterations in the diffusing capacity of the lung for carbon monoxide (DLCO) (e.g., improved gas exchange) in conjunction with the determination of the level of smoking cessation by means of serum cotinine levels. As per standard protocol, the research team was blinded to treatment group until the subject had exited the trial and DECT image analysis was completed. Importantly, because serum cotinine levels were used to determine smoking cessation status, subjects were not allowed to use nicotine replacement therapy. However, in order to encourage the likelihood of smoking cessation, subjects were given incentive-based programming.

In this paradigm, subjects are financially rewarded for smoking cessation (see, for example, Alford et al., 2010, PNAS USA, 107:7485-90; Iyer et al., 2016, Am. J. Resp. Crit. Care Med., 193:652-61). This paradigm has been highly effective in increasing smoking cessation in those receiving other smoking interventions including bupropion or nicotine replacement therapy. Hence, it was reasonable to assume the use of this behavioral intervention would increase the overall rate of smoking cessation and provide enough ex-smokers to determine whether sildenafil is effective in aiding tissue re-oxygenation and reduction in parenchymal inflammation.

The results were analyzed from the first 48 subjects who enrolled in the study. Briefly, the subjects in the cohort were largely of European ancestry, and the cohort was evenly balanced between males and females, with subjects of both genders tending to be between 30 and 60 years of age. As evidenced by their pre-experiment serum cotinine levels, they also tended to be moderate to heavy smokers. See Table 2.

TABLE 2
Clinical Demographics of First 48 Subjects
Participating in Sildenafil Trial

| | |
|---|---|
| Male | Female |

| | | |
|---|---|---|
| N | 24 | 23* |

| | | | | |
|---|---|---|---|---|
| Age | 43 ± 12 | years | 44 ± 9 | years |
| Cotinine | 234 ± 94 | ng/ml | 258 ± 110 | ng/ml |

*Data from one subject who dropped out immediately after enrolling is not available.

In order to qualify for the financial incentive of $400, the subjects had to complete all 4 visits (intake and 3 follow-up visits at 30, 60 and 90 days) and had to quit smoking as evidenced by serum cotinine levels. In total, of the first 48 subjects enrolled in the study, 9 subjects completed just the intake, 7 subjects participated in both the intake and first visit, and 31 subjects completed all 4 visits in the study (Table 3). Data from one subject who dropped immediately from the study is not available.

TABLE 3
Outcomes of Clinical Intervention

| Treatment | Placebo | Sildenafil |
|---|---|---|
| N | 24 | 24 |
| Visits Completed | | |
| One | 6 | 3 |
| Two | 4 | 2 |
| Three | 0 | 1 |
| Four | 13 | 18 |
| Cessation | | |
| Quit at 90 days | 7 | 12 |
| <50% reduction of cotinine | 0 | 3 |
| Visits with <2 ng/ml cotinine | 19 | 36 |

*Data from one subject who dropped out immediately after enrolling is not available

Analysis of the data using an intention to treat approach provides evidence that the use of sildenafil in this treatment paradigm was associated with decreased smoking. With regards to complete cessation in the 31 subjects who completed all four visits, there was a trend for the sildenafil group to have more success vs. placebo: (7 of 24 vs. 12 of 24, p<0.09, Exact Test). When considering either a full or partial response of treatment (≥average 50% reduction of intake cotinine levels compared to average of 30-, 60- and 90-day visits), there was a significant effect of sildenafil vs. placebo: (7 of 24 vs. 15 of 24, p<0.02 Exact Test). Finally, and perhaps critically, the subjects in the sildenafil group not only participated in more visits (67 vs. 56 visits out a total possible visit count of 96 visits), but they were more likely to be abstinent during the follow-up visit. At the 30-, 60- and 90-day visits, the sildenafil-treated subjects were more likely to have negative serum cotinine values (<2 ng/ml) than the placebo group (19 of 72 potential visits vs. 36 of 72 potential visits, p<0.002, Exact Test).

A natural question, given the well-known properties of sildenafil as agent for the treatment of erectile dysfunction, is whether males responded preferentially. However, there was no detectable difference with 6 males and 6 females having achieved abstinence at 90 days as evidenced by negative serum cotinine determinations. Additionally, the 20 mg per day dose of sildenafil used in this study was well below the 50-100 mg per day starting dose recommended for erectile dysfunction.

There was a significant loss of lung density (inflammation) coupled with an improvement in DLCO in the group who successfully stopped smoking for 90 days on sildenafil with no significant change in the other groups (placebo with and without smoking cessation and sildenafil without smoking cessation). In summary, in this pilot trial that included incentive-based programming, sildenafil was significantly associated with several key smoking cessation outcomes.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of reducing an individual's desire to smoke and/or frequency of smoking, comprising:
administering to the individual a phosphodiesterase-5 (PDE5) inhibitor or pharmaceutically acceptable salt thereof in an amount effective to reduce the individual's desire to smoke, reduce the frequency of smoking, or both.

2. The method of claim 1 wherein the PDE5 inhibitor is a compound of Formula I: 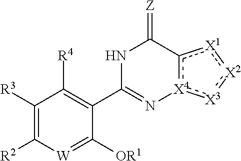
or a pharmaceutically acceptable salt thereof, wherein:
Z is O or S;
W is N or CR⁵;
X¹ and X² are each independently selected from N, NR⁶, and CR⁷;
X³ is N or CR⁸;
X⁴ is C or N;
R¹ is H, C₁₋₆ alkyl, or -L-O—(C₁₋₆ alkyl);
R² is H, C₁₋₆ alkyl, or C₄₋₁₀ cycloalkyl;
R³ is H, NO₂, C₁₋₆ alkyl, C₄₋₁₀ cycloalkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;
R⁴ is H, C₁₋₆ alkyl, C₄₋₁₀ cycloalkyl, or C(═O)(C₁₋₆ alkyl);
R⁵ is H, C₁₋₆ alkyl, or C₄₋₁₀ cycloalkyl;
R⁶ is H, C₁₋₆ alkyl, C₄₋₁₀ cycloalkyl, -L-O—(C₁₋₆ alkyl), -L-O—(C₄₋₁₀ cycloalkyl), -L-aryl, -L-hetAr¹, or -L-hetCyc¹;
R⁷ is H, C₁₋₆ alkyl, or C₄₋₁₀ cycloalkyl;
R⁸ is H, C₁₋₆ alkyl, or C₄₋₁₀ cycloalkyl;
R⁹ is C₁₋₆ alkyl, C₄₋₁₀ cycloalkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;
R¹⁰ is H or C₁₋₆ alkyl;
R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;
hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl);
L is absent or C₁₋₆ alkyl; and
the dashed lines can be single or double bonds.

3. The method of claim 2, wherein the compound of Formula I is a compound of Formula Ia: 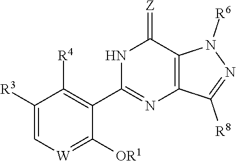
or a pharmaceutically acceptable salt thereof, wherein:
Z is O or S;
W is N or CR⁵;
R¹ is H, C₁₋₆ alkyl, or -L-O—(C₁₋₆ alkyl);
R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;
R⁴ is H, C₁₋₆ alkyl, or C(═O)(C₁₋₆ alkyl);
R⁵ is H or C₁₋₆ alkyl;
R⁶ is H, C₁₋₆ alkyl, -L-O—(C₁₋₆ alkyl), -L-aryl, -L-hetAr¹, or -L-hetCyc¹;
R⁸ is H or C₁₋₆ alkyl;
R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;
R¹⁰ is H or C₁₋₆ alkyl;
R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;
hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and
L is absent or C₁₋₆ alkyl.

4. The method of claim 3, wherein Z is O.

5. The method of claim 3, wherein Z is S.

6. The method of any one of claims 3-5, wherein W is CH.

7. The method of any one of claims 3-6, wherein R¹ is H.

8. The method of any one of claims 3-6, wherein R¹ is C₁₋₃ alkyl.

9. The method of claim 8, wherein R¹ is ethyl.

10. The method of claim 8, wherein R¹ is propyl.

11. The method of any one of claims 3-10, wherein R⁴ is H.

12. The method of any one of claims 3-11, wherein R⁶ is C₁₋₃ alkyl.

13. The method of claim 12, wherein R⁶ is methyl.

14. The method of any one of claims 3-13, wherein R⁸ is C₁₋₃ alkyl.

15. The method of claim 14, wherein R⁸ is propyl.

16. The method of claim 2, wherein the compound of Formula I is a compound of Formula Ib: 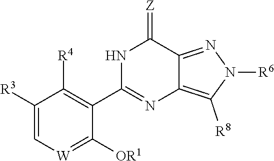
or a pharmaceutically acceptable salt thereof, wherein:
Z is O or S;
W is N or CR⁵;
R¹ is H, C₁₋₆ alkyl, or -L-O—(C₁₋₆ alkyl);
R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;
R⁴ is H, C₁₋₆ alkyl, or C(═O)(C₁₋₆ alkyl);
R⁵ is H or C₁₋₆ alkyl;
R⁶ is H, C₁₋₆ alkyl, -L-O—(C₁₋₆ alkyl), -L-aryl, -L-hetAr¹, or -L-hetCyc¹;
R⁸ is H or C₁₋₆ alkyl;
R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;
R¹⁰ is H or C₁₋₆ alkyl;
R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;
hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and
L is absent or C₁₋₆ alkyl.

17. The method of claim 16, wherein Z is O.

18. The method of claim 16 or 17, wherein W is CH.

19. The method of claim 16 or 17, wherein W is N.

20. The method of any one of claims 16-19, wherein R¹ is C₁₋₆ alkyl.

21. The method of claim 20, wherein R¹ is ethyl, propyl, or butyl.

22. The method of any one of claims 16-19, wherein R¹ is —(C₁₋₆ alkyl)-O—(C₁₋₆ alkyl).

23. The method of any one of claims 16-22, wherein R⁴ is H.

24. The method of any one of claims 16-22, wherein R⁴ is C(═O)(C₁₋₆ alkyl).

25. The method of claim 24, wherein C₁₋₆ alkyl is methyl.

26. The method of any one of claims 16-25, wherein R⁶ is C₁₋₃ alkyl.

27. The method of any one of claims 16-25, wherein R⁶ is —(C₁₋₆ alkyl)-O—(C₁₋₆ alkyl).

28. The method of any one of claims 16-25, wherein R⁶ is phenyl.

29. The method of any one of claims 16-25, wherein R⁶ is —(C₁₋₆ alkyl)-hetAr¹.

30. The method of claim 29, wherein hetAr¹ is pyridine.

31. The method of any one of claims 16-25, wherein R⁶ is hetCyc¹ optionally substituted with C₁₋₆ alkyl.

32. The method of claim 31, wherein R⁶ is piperidine or azetidine substituted with C₁₋₃ alkyl.

33. The method of any one of claims 16-32, wherein R⁸ is C₁₋₃ alkyl.

34. The method of claim 33, wherein R⁸ is ethyl.

35. The method of claim 2, wherein the compound of Formula I is a compound of Formula Ic: 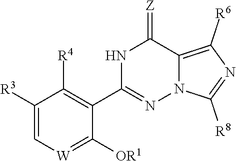
or a pharmaceutically acceptable salt thereof, wherein:
Z is O or S;
W is N or CR⁵;
R¹ is H, C₁₋₆ alkyl, or -L-O—(C₁₋₆ alkyl);
R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;
R⁴ is H, C₁₋₆ alkyl, or C(═O)(C₁₋₆ alkyl);
R⁵ is H or C₁₋₆ alkyl;
R⁶ is H, C₁₋₆ alkyl, -L-O—(C₁₋₆ alkyl), -L-aryl, -L-hetAr¹, or -L-hetCyc¹;
R⁸ is H or C₁₋₆ alkyl;
R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;
R¹⁰ is H or C₁₋₆ alkyl;
R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;
hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and
L is absent or C₁₋₆ alkyl.

36. The method of claim 35, wherein Z is O.

37. The method of claim 35 or 36, wherein W is CH.

38. The method of any one of claims 35-37, wherein R¹ is C₁₋₃ alkyl.

39. The method of claim 38, wherein R¹ is ethyl.

40. The method of any one of claims 35-39, wherein R⁴ is H.

41. The method of any one of claims 35-40, wherein R⁶ is C₁₋₃ alkyl.

42. The method of claim 41, wherein R⁶ is methyl.

43. The method of any one of claims 35-42, wherein R⁸ is C₁₋₃ alkyl.

44. The method of claim 43, wherein R⁸ is propyl.

45. The method of claim 2, wherein the compound of Formula I is a compound of Formula Id: 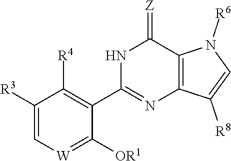
or a pharmaceutically acceptable salt thereof, wherein:
Z is O or S;
W is N or CR⁵;
R¹ is H, C₁₋₆ alkyl, or -L-O—(C₁₋₆ alkyl);
R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;
R⁴ is H, C₁₋₆ alkyl, or C(═O)(C₁₋₆ alkyl);
R⁵ is H or C₁₋₆ alkyl;
R⁶ is H, C₁₋₆ alkyl, -L-O—(C₁₋₆ alkyl), -L-aryl, -L-hetAr¹, or -L-hetCyc¹;
R⁸ is H or C₁₋₆ alkyl;
R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;
R¹⁰ is H or C₁₋₆ alkyl;
R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;
hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and
L is absent or C₁₋₆ alkyl.

46. The method of claim 45, wherein Z is O.

47. The method of claim 45 or 46, wherein W is CH.

48. The method of any one of claims 45-47, wherein R¹ is C₁₋₃ alkyl.

49. The method of claim 48, wherein R¹ is propyl.

50. The method of any one of claims 45-49, wherein R⁴ is H.

51. The method of any one of claims 45-50, wherein R⁶ is C₁₋₃ alkyl.

52. The method of claim 51, wherein R⁶ is ethyl.

53. The method of any one of claims 45-52, wherein R⁸ is C1-3 alkyl.

54. The method of claim 53, wherein R⁸ is propyl.

55. The method of claim 2, wherein the compound of Formula I is a compound of Formula Ie: 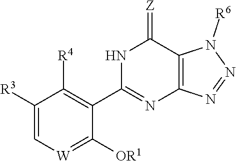
or a pharmaceutically acceptable salt thereof, wherein:
Z is O or S;
W is N or CR⁵;
R¹ is H, C₁₋₆ alkyl, or -L-O—(C₁₋₆ alkyl);
R³ is H, NO₂, C₁₋₆ alkyl, C₁₋₆ alkyl(hetCyc¹), C(═O)R⁹, SO₂(hetCyc¹), or SO₂NR¹⁰R¹¹, wherein any C₁₋₆ alkyl is optionally substituted with hydroxy or halogen;
R⁴ is H, C₁₋₆ alkyl, or C(═O)(C₁₋₆ alkyl);
R⁵ is H or C₁₋₆ alkyl;
R⁶ is H, C₁₋₆ alkyl, -L-O—(C₁₋₆ alkyl), -L-aryl, -L-hetAr¹, or -L-hetCyc¹;
R⁹ is C₁₋₆ alkyl, C₁₋₆ alkyl(hetCyc¹), hetCyc¹, or C₁₋₆ alkyl(hetCyc¹)(C₂₋₆ alkenyl)(aryl), wherein any C₁₋₆ alkyl is optionally substituted with hydroxy or halogen;
R¹⁰ is H or C₁₋₆ alkyl;
R¹¹ is H, C₁₋₆ alkyl, C₁₋₆ alkyl(NR′R″), C₁₋₆ alkyl(hetCyc¹), and (C₁₋₆ alkyl)C(═O)NR′(C₁₋₆ alkyl)C(═O)NR′R″, wherein R′ and R″ are each independently selected from H and C₁₋₆ alkyl;
hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy;
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and
L is absent or C₁₋₆ alkyl.

56. The method of claim 55, wherein Z is O.

57. The method of claim 55 or 56, wherein W is CH.

58. The method of any one of claims 55-57, wherein R¹ is C₁₋₃ alkyl.

59. The method of claim 58, wherein R¹ is propyl.

60. The method of any one of claims 55-59, wherein R⁴ is H.

61. The method of any one of claims 55-60, wherein R⁶ is H.

62. The method of any one of claims 2-61, wherein R³ is selected from

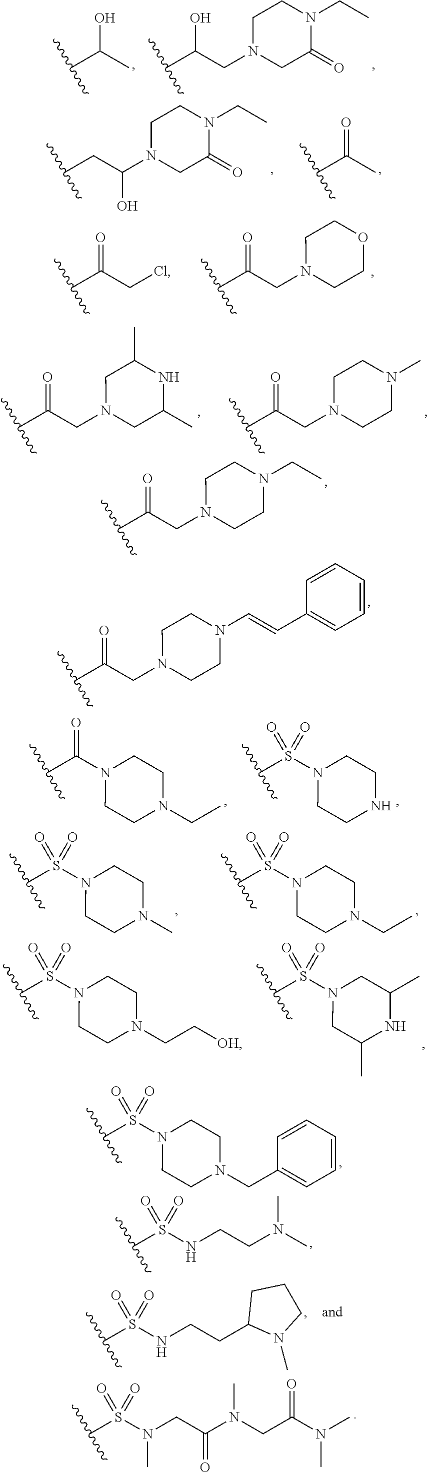

63. The method of claim 1, wherein the PDE5 inhibitor is a compound of Formula II: 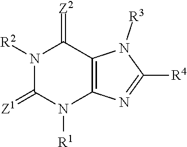
or a pharmaceutically acceptable salt thereof, wherein:
Z¹ is O or S;
Z² is O or S;
R¹ is H, C₁₋₆ alkyl, C₄₋₁₀ cycloalkyl, or C₁₋₆ hydroxyalkyl;
R² is H, C₁₋₆ alkyl, C₄₋₁₀ cycloalkyl, or C₁₋₆ hydroxyalkyl;
R³ is H, C₁₋₆ alkyl, C₄₋₁₀ cycloalkyl, C₁₋₆ alkyl(hetCyc¹), C₁₋₆ alkyl(hetAr¹), or C₁₋₆ alkyl(aryl), wherein any C₁₋₆ alkyl is optionally substituted with one or more hydroxy and halogen, and aryl is optionally substituted with one or more substituents independently selected from halogen, C₁₋₆ alkyl, amino, cyano, C₁₋₆ alkoxy, and hydroxy;
R⁴ is H, C₁₋₆ alkyl, C₄₋₁₀ cycloalkyl, or NR′R″, wherein R′ and R″ are each independently selected from H, C₁₋₆ alkyl, C₄₋₁₀ cycloalkyl, hetCyc¹, hetAr¹, aryl, C₁₋₆ alkyl(hetCyc¹), C₁₋₆ alkyl(hetAr¹), and C₁₋₆ alkyl(aryl), and wherein any C₁₋₆ alkyl or C₄₋₁₀ cycloalkyl is optionally substituted with one or more hydroxy and halogen, and aryl is optionally substituted with one or more substituents independently selected from halogen, C₁₋₆ alkyl, amino, cyano, C₁₋₆ alkoxy, and hydroxy;
hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy; and
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

64. The method of claim 63, wherein Z¹ and Z² are each O.

65. The method of claim 63 or 64, wherein R¹ is C₁₋₃ hydroxyalkyl.

66. The method of any one of claims 63-65, wherein R² is C₁₋₃ alkyl.

67. The method of claim 66, wherein R² is ethyl.

68. The method of any one of claims 63-67, wherein R³ is C₁₋₃ alkyl(aryl) optionally substituted with one or two substituents independently selected from halogen and C₁₋₆ alkoxy.

69. The method of any one of claims 63-68, wherein R⁴ is NR′R″.

70. The method of claim 69, wherein R′ is H.

71. The method of claim 69 or 70, wherein R″ is C₄₋₁₀ cycloalkyl optionally substituted with hydroxy.

72. The method of claim 71, wherein C₄₋₁₀ cycloalkyl is cyclopentyl.

73. The method of claim 1, wherein the PDE5 inhibitor is a compound of Formula III: 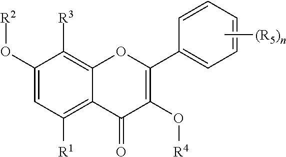
or a pharmaceutically acceptable salt thereof, wherein:
R¹ is H, hydroxy, C₁₋₆ alkyl, C₄₋₁₀ cycloalkyl, or C₁₋₆ hydroxyalkyl;
R² is H, C₁₋₆ alkyl, C₄₋₁₀ cycloalkyl, or hetCyc¹;
R³ is H, C₁₋₆ alkyl, C₄₋₁₀ cycloalkyl, or C₂₋₁₀ alkenyl;
R⁴ is H, C₁₋₆ alkyl, C₄₋₁₀ cycloalkyl, or hetCyc¹;
R⁵ is H, C₁₋₆ alkyl, C₄₋₁₀ cycloalkyl, or C₁₋₆ alkoxy;
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and
n is 0 to 5.

74. The method of claim 73, wherein R¹ is hydroxy.

75. The method of claim 73 or 74, wherein R² is hetCyc¹ optionally substituted with one to four substituents independently selected from hydroxy and C₁₋₃ hydroxyalkyl.

76. The method of claim 75, wherein R² is tetrahydropyran substituted with one to four substituents independently selected from hydroxy and C₁₋₃ hydroxyalkyl.

77. The method of any one of claims 73-76, wherein R³ is C₂₋₁₀ alkenyl.

78. The method of claim 77, wherein R³ is C₅ alkenyl.

79. The method of any one of claims 73-78, wherein R⁴ is hetCyc¹ optionally substituted with one to four substituents independently selected from hydroxy and C₁₋₃ alkyl.

80. The method of claim 79, wherein R⁴ is tetrahydropyran substituted with one to four substituents independently selected from hydroxy and C₁₋₃ alkyl.

81. The method of any one of claims 73-80, wherein R⁵ is C₁₋₆ alkoxy.

82. The method of claim 81, wherein R⁵ is methoxy.

83. The method of any one of claims 73-82, wherein n is 1.

84. The method of claim 1, wherein the PDE5 inhibitor is a compound of Formula IV: 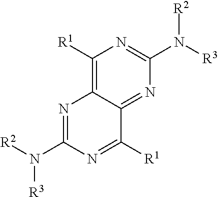
or a pharmaceutically acceptable salt thereof, wherein:
R¹ is H, amino, C₁₋₆ alkyl, C₁₋₆ hydroxyalkyl, C₁₋₆ alkoxy, C₄₋₁₀ cycloalkyl, or hetCyc¹;
R² and R³ are each independently selected from H, C₁₋₆ alkyl, C₁₋₆ hydroxyalkyl, C₁₋₆ alkoxy, C₄₋₁₀ cycloalkyl, and hetCyc¹; and
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

85. The method of claim 84, wherein R¹ is hetCyc¹.

86. The method of claim 85, wherein hetCyc¹ is piperidine.

87. The method of any one of claims 84-86, wherein R² is C₁₋₃ hydroxyalkyl.

88. The method of any one of claims 84-87, wherein R³ is C₁₋₃ hydroxyalkyl.

89. The method of any one of claims 84-88, wherein R² and R³ are each C₁₋₃ hydroxyalkyl.

90. The method of claim 1, wherein the PDE5 inhibitor is a compound of Formula V: 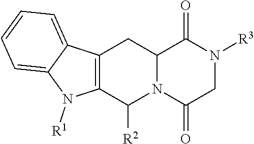
or a pharmaceutically acceptable salt thereof, wherein:
R¹, R², and R³ are each independently selected from H, C₁₋₆ alkyl, C₁₋₆ hydroxyalkyl, C₁₋₆ alkoxy, C₄₋₁₀ cycloalkyl, and hetCyc¹; and
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl).

91. The method of claim 90, wherein R¹ is H.

92. The method of claim 90 or 91, wherein R² is hetCyc¹.

93. The method of claim 92, wherein hetCyc¹ is 1,3-benzodioxole.

94. The method of any one of claims 90-93, wherein R³ is C₁₋₃ alkyl.

95. The method of claim 95, wherein R³ is methyl.

96. The method of claim 1, wherein the PDE5 inhibitor is a compound of Formula VI: 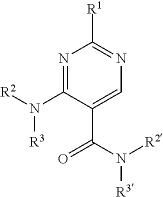
or a pharmaceutically acceptable salt thereof, wherein:
R¹ is H, C₁₋₆ alkyl, C₁₋₆ hydroxyalkyl, C₁₋₆ alkoxy, C₄₋₁₀ cycloalkyl, or hetCyc¹;
R², R^{2′}, R³ and R^{3′} are each independently selected from H, C₁₋₆ alkyl, C₁₋₆ hydroxyalkyl, C₁₋₆ alkoxy, C₄₋₁₀ cycloalkyl, aryl, hetCyc¹, hetAr¹, C₁₋₆ alkyl(aryl), C₁₋₆ alkyl(hetCyc¹), and C₁₋₆ alkyl(hetAr¹), wherein aryl is optionally substituted with halogen, C₁₋₆ alkyl, C₁₋₆ alkoxy, amino, cyano, hydroxy, and C₁₋₆ hydroxyalkyl;
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl); and
hetAr¹ is a 5-12 membered heteroaryl ring having 1-3 ring atoms independently selected from N, O, and S which is optionally substituted with one or more substituents independently selected from halogen, C₁₋₆ alkyl, amino, cyano, C₁₋₆ alkoxy, and hydroxy.

97. The method of claim 96, wherein R¹ is hetCyc¹ optionally substituted with C₁₋₃ hydroxyalkyl.

98. The method of claim 97, wherein hetCyc¹ is pyrrolidine.

99. The method of any one of claim 96-98, wherein R² and R^{2′} are each H.

100. The method of any one of claims 96-99, wherein R³ is C₁₋₃ alkyl(aryl) substituted with one or two substituents selected from halogen and C₁₋₃ alkoxy.

101. The method of any one of claims 96-100, wherein R^{3′} is C₁₋₃ alkyl(hetAr¹).

102. The method of claim 101, wherein hetAr¹ is pyrimidine.

103. The method of claim 1, wherein the PDE5 inhibitor is a compound of Formula VII: 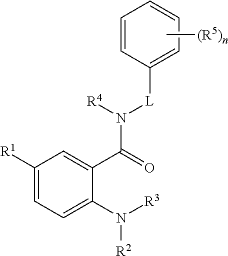
or a pharmaceutically acceptable salt thereof, wherein:
R¹ is H, amino, nitro, C₁₋₆ alkyl, C₁₋₆ hydroxyalkyl, C₁₋₆ alkoxy, C₄₋₁₀ cycloalkyl, or hetCyc¹;
R², R³, and R⁴ are each independently selected from H, C₁₋₆ alkyl, C₁₋₆ hydroxyalkyl, C₁₋₆ alkoxy, C₄₋₁₀ cycloalkyl, and hetCyc¹;
R⁵ is H, amino, nitro, C₁₋₆ alkyl, C₁₋₆ hydroxyalkyl, C₁₋₆ alkoxy, C₄₋₁₀ cycloalkyl;
hetCyc¹ is a 6-10 membered heterocycloalkyl ring system having 1-3 ring atoms independently selected from N, O, and S which is optionally substituted with one or more substituents independently selected from halogen, C₁₋₆ alkyl, oxo, amino, cyano, C₁₋₆ alkoxy, hydroxy, C₁₋₆ hydroxyalkyl, and C₁₋₆ alkyl(aryl);
L is —C₁₋₆ alkyl- or —C₁₋₆ alkoxy-; and
n is 0 to 5.

104. The method of claim 103, wherein R¹ is nitro.

105. The method of claim 103 or 104, wherein R² is H.

106. The method of any one of claims 103-105, wherein R³ is C₁₋₃ hydroxyalkyl.

107. The method of any one of claims 103-106, wherein R⁴ is H.

108. The method of any one of claims 103-107, wherein R⁵ is C₁₋₆ alkoxy.

109. The method of claim 108, wherein R⁵ is methoxy.

110. The method of any one of claims 103-109, wherein n is 1.

111. The method of any one of claims 103-110, wherein L is —C₁₋₃ alkyl-.

112. The method of any one of claims 1-111, wherein the PDE5 inhibitor is selected from 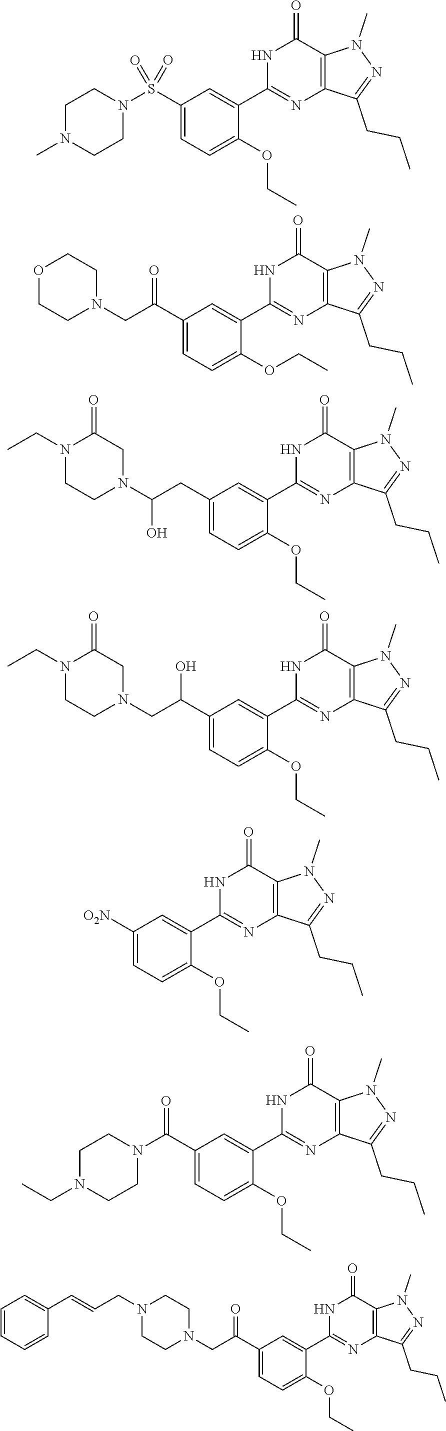 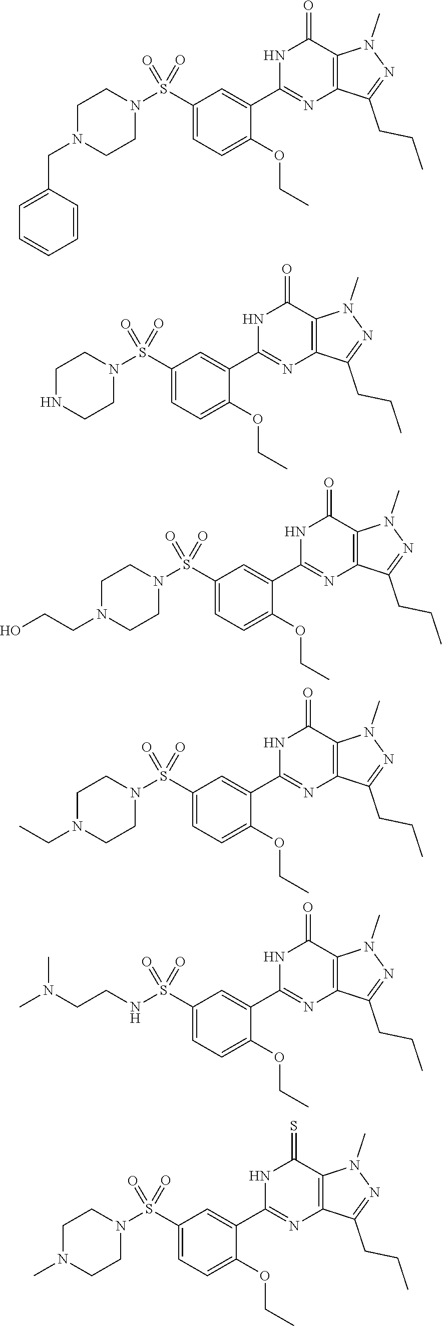 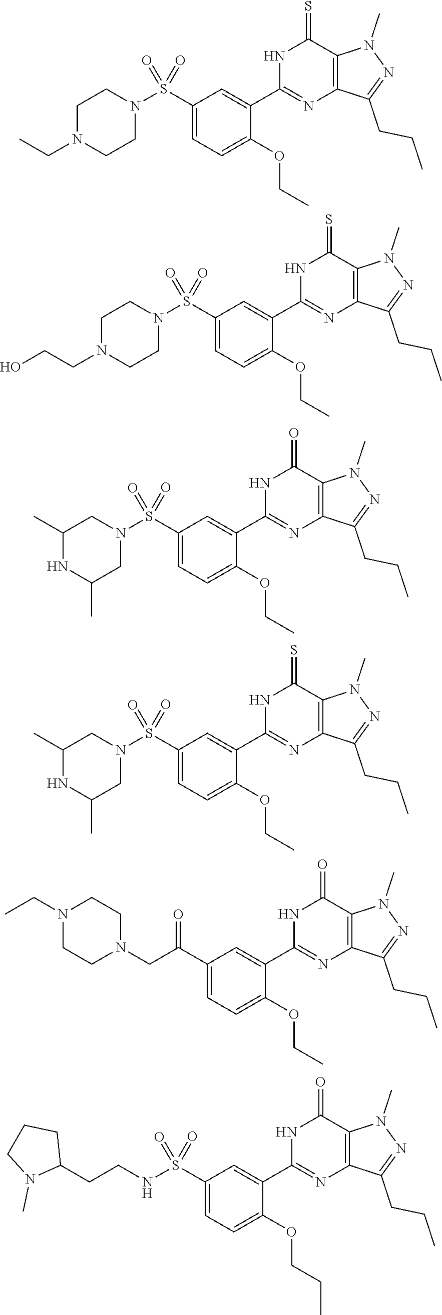 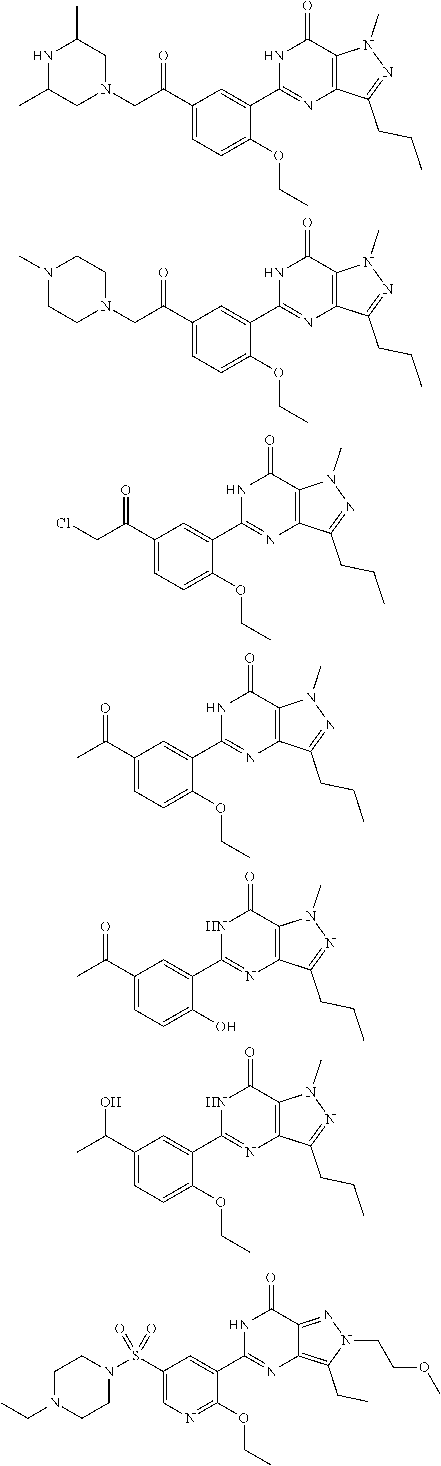 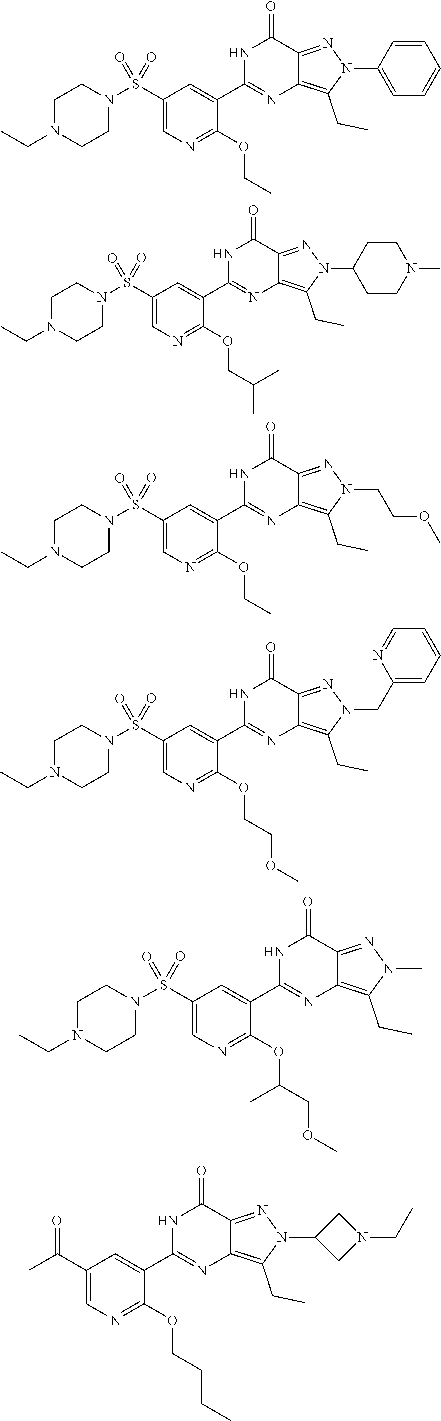 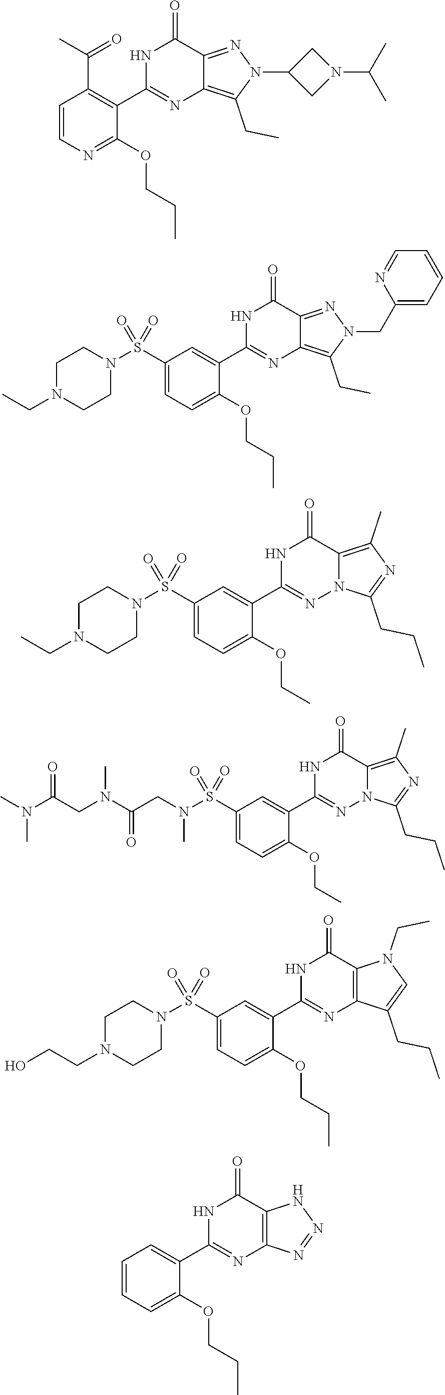 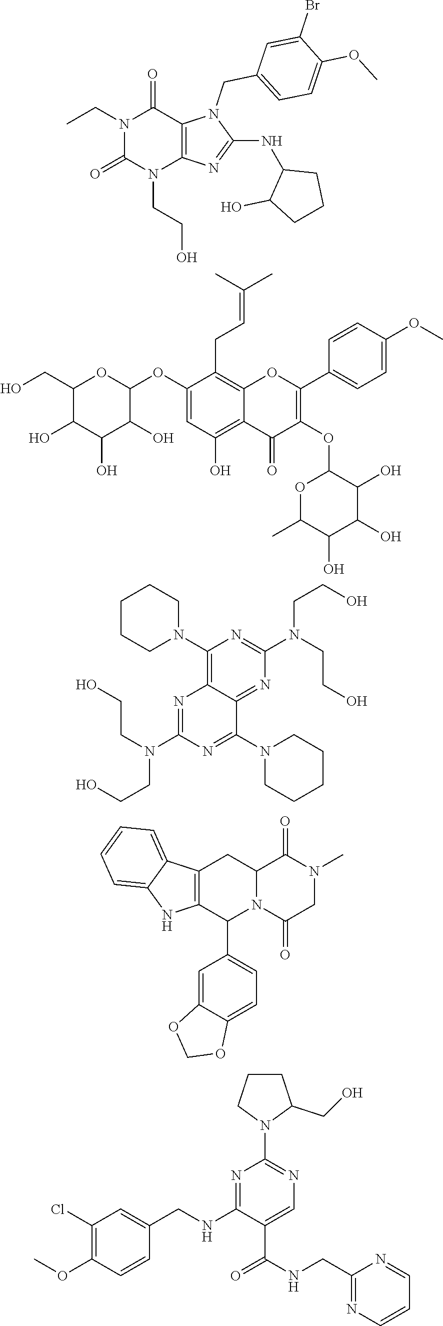 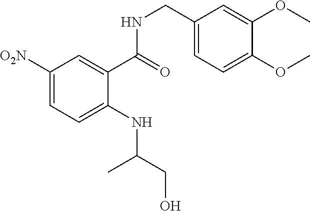 [SMILES: COc1ccc(CNC(=O)c2cc([N+](=O)[O-])ccc2NC(C)CO)cc1OC]
and pharmaceutically acceptable salts thereof.

113. The method of claim 1, wherein the PDE5 inhibitor is selected from 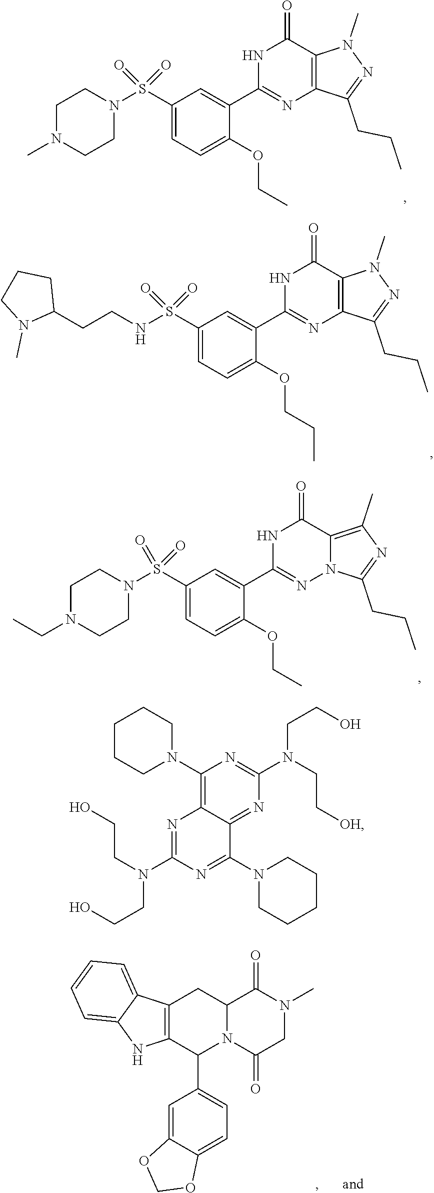 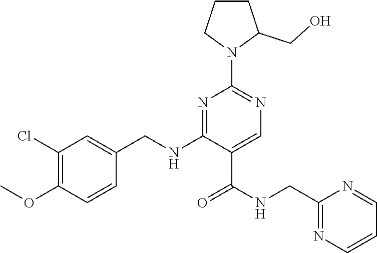 [SMILES: COc1ccc(CNc2nc(N3CCCC3CO)ncc2C(=O)NCc2ncccn2)cc1Cl]
and pharmaceutically acceptable salts thereof.

114. The method of claim 1, wherein the PDE5 inhibitor is sildenafil: 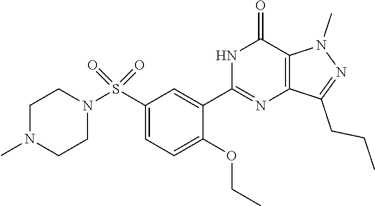 [SMILES: CCCc1nn(C)c2c(=O)[nH]c(-c3cc(S(=O)(=O)N4CCN(C)CC4)ccc3OCC)nc12]
or a pharmaceutically acceptable salt thereof.

115. The method of any one of claims 1-114, wherein the PDE5 inhibitor or pharmaceutically acceptable salt thereof is administered to the individual at a dose of about 1 mg/day to about 150 mg/day.

116. The method of any one of claims 1-115, wherein the effective amount of the PDE5 inhibitor or pharmaceutically acceptable salt thereof is from about 1 mg/day to about 150 mg/day.

117. The method of any one of claims 1-116, wherein the PDE5 inhibitor or pharmaceutically acceptable salt thereof is formulated for oral administration.

118. The method of any one of claims 1-117, wherein the PDE5 inhibitor or pharmaceutically acceptable salt thereof is formulated for extended/delayed release.

119. The method of any one of claims 1-118, further comprising co-administering to the individual nicotine replacement (e.g., in the form of gum or a transdermal patch), bupropion (e.g., Wellbutrin®), tadalafil (e.g., Cialis®), vardenafil (e.g., Levitra®), or varenicline (e.g., Chantix®), or derivatives, enantiomers, metabolites, or pharmaceutically acceptable salts thereof.

120. The method of any one of claims 1-119, further comprising applying motivational interviewing, incentive based programming, or other psychological technique(s) to the individual.

121. The method of any one of claims 1-120, wherein the individual, following administration for a period of time, exhibits an improvement in the lung diffusing capacity for carbon monoxide (DLCO).

122. The method of any one of claims 1-121, wherein the individual, following administration for a period of time, exhibits tissue re-perfusion.

123. The method of any one of claims 1-122, wherein the individual, following administration for a period of time, exhibits a reduction in parenchymal inflammation (or lung density).

124. An article of manufacture, comprising:
at least one dose of a PDE5 inhibitor of any one of claims 2-114 or a pharmaceutically acceptable salt thereof in an amount effective to reduce an individual's desire to smoke and/or frequency of smoking; and
at least one dose of a nicotine replacement (e.g., in the form of gum or a transdermal patch), bupropion (e.g., Wellbutrin®) tadalafil (e.g., Cialis®), vardenafil (e.g., Levitra®), or varenicline (e.g., Chantix®), or derivatives, enantiomers, metabolites, or pharmaceutically acceptable salts thereof in an amount effective to reduce an individual's desire to smoke and/or frequency of smoking.

125. The method of claim 124, further comprising applying motivational interviewing, incentive based programming, or other psychological technique(s) to the individual.

126. A method of reducing an individual's desire to smoke and/or frequency of smoking, comprising:
administering to the individual an amount of sildenafil (Viagra®), a derivative of sildenafil, an enantiomer of sildenafil, an active metabolite of sildenafil, or a pharmaceutically acceptable salt of sildenafil effective to reduce the individual's desire to smoke and/or frequency of smoking.

127. The method of claim 126, wherein the sildenafil or derivative, enantiomer or pharmaceutically acceptable salt thereof is administered to the individual at a dose of about 1 mg/day to about 150 mg/day.

128. The method of claim 126, wherein the effective amount of the sildenafil or derivative, enantiomer, metabolite, or pharmaceutically acceptable salt thereof is from about 1 mg/day to about 150 mg/day.

129. The method of any one of claims 126-128, wherein the sildenafil or derivative, enantiomer, metabolite, or pharmaceutically acceptable salt thereof is formulated for oral administration.

130. The method of any one of claims 126-129, wherein the sildenafil or derivative, enantiomer, metabolite, or pharmaceutically acceptable salt thereof is formulated for extended/delayed release.

131. The method of any one of claims 126-130, further comprising co-administering to the individual a nicotine replacement (e.g., in the form of gum or a transdermal patch), bupropion (e.g., Wellbutrin®), tadalafil (e.g., Cialis®), vardenafil (e.g., Levitra®), or varenicline (e.g., Chantix®), or derivatives, enantiomers, metabolites, or pharmaceutically acceptable salts thereof.

132. The method of any one of claims 126-131, further comprising applying motivational interviewing, incentive based programming, or other psychological technique(s) to the individual.

133. The method of any one of claims 126-132, wherein the individual, following administration for a period of time, exhibits an improvement in the lung diffusing capacity for carbon monoxide (DLCO).

134. The method of any one of claims 126-133, wherein the individual, following administration for a period of time, exhibits tissue re-perfusion.

135. The method of any one of claims 126-134, wherein the individual, following administration for a period of time, exhibits a reduction in parenchymal inflammation (or lung density).

136. An article of manufacture, comprising:
at least one dose of sildenafil (Viagra®), a derivative of sildenafil, an enantiomer of sildenafil, an active metabolite of sildenafil, or a pharmaceutically acceptable salt of sildenafil effective to reduce an individual's desire to smoke and/or frequency of smoking; and
at least one dose of a nicotine replacement (e.g., in the form of gum or a transdermal patch), bupropion (e.g., Wellbutrin®) tadalafil (e.g., Cialis®), vardenafil (e.g., Levitra®), or varenicline (e.g., Chantix®), or derivatives, enantiomers, metabolites, or pharmaceutically acceptable salts thereof effective to reduce an individual's desire to smoke and/or frequency of smoking.

137. The article of manufacture of claim 136, wherein the at least one dose of the sildenafil or derivative, enantiomer, metabolite, or pharmaceutically acceptable salt thereof comprises about 1 mg to about 150 mg.

138. The article of manufacture of claim 136 or claim 137, wherein the sildenafil or derivative, enantiomer, metabolite, or pharmaceutically acceptable salt thereof is formulated for oral administration.

139. The article of manufacture of any one of claims 136-138, wherein the sildenafil or derivative, enantiomer, metabolite, or pharmaceutically acceptable salt thereof is formulated for extended/delayed release.

140. An article of manufacture, comprising at least one dose of sildenafil (Viagra®), a derivative of sildenafil, an enantiomer of sildenafil, an active metabolite of sildenafil, or a pharmaceutically acceptable salt of sildenafil, wherein the at least one dose comprises about 1 mg to about 40 mg of the sildenafil or derivative, enantiomer, metabolite, or pharmaceutically acceptable salt thereof.

141. The article of manufacture of claim 140, further comprising at least one dose of a nicotine replacement (e.g., in the form of gum or a transdermal patch), bupropion (e.g., Wellbutrin®) tadalafil (e.g., Cialis®), vardenafil (e.g., Levitra®), or varenicline (e.g., Chantix®), or derivatives, enantiomers, metabolites, or pharmaceutically acceptable salts thereof.

142. The article of manufacture of claim 140 or 141, wherein the sildenafil or derivative, enantiomer, metabolite, or pharmaceutically acceptable salt thereof is formulated for oral administration.

143. The article of manufacture of any one of claims 140-142, wherein the sildenafil or derivative, enantiomer, metabolite, or pharmaceutically acceptable salt thereof is formulated for extended/delayed release.
